(19) United States
(12) Patent Application Publication
(10) Pub. No.: US 2026/0216174 A1
(43) Pub. Date: Jul. 30, 2026

(54) TREATMENT OF ER+ BREAST CANCER COMPRISING HOMOLOGOUS RECOMBINATION DEFICIENCY USING PARG INHIBITOR

(71) Applicant: IDEAYA BIOSCIENCES, INC., South San Francisco, CA (US)

(72) Inventors: Monah ABED, South San Francisco, CA (US); Diana Marcela MUNOZ DELGADO, South San Francisco, CA (US); Mark LACKNER, South San Francisco, CA (US); Zineb MOUNIR, South San Francisco, CA (US); Vidya SESHADRI, South San Francisco, CA (US)

(21) Appl. No.: 19/145,667

(22) PCT Filed: Jan. 5, 2024
(86) PCT No.: PCT/US2024/010508

§ 371 Date: Jul. 3, 2025

Related U.S. Application Data
(60) Provisional application No. 63/478,871, filed on Jan. 6, 2023.
(60) Provisional application No. 63/605,118, filed on Dec. 1, 2023.

(51) Int. Cl. A61K 31/496; A61P 35/00

(52) U.S. Cl. CPC A61K 31/496; A61P 35/00

(57) ABSTRACT

The present disclosure concerns new methods of treating cancer, e.g., breast cancer, in a patient in need thereof, comprising, inter alia, the use of poly(ADP-ribose) glycohydrolase (PARG) modulators, or pharmaceutically acceptable salts thereof.

### PRIORITY CLAIM

This application claims the benefit of priority under 35 U.S.C § 119 (e) to U.S. Provisional Application Ser. No. 63/478,871 filed Jan. 6, 2023 and U.S. Provisional Application Ser. No. 63/605,118 filed Dec. 1, 2023, the disclosure of each is incorporated herein by reference in their entirety.

### TECHNICAL FIELD

The present disclosure provides methods of treating cancer, e.g., breast cancer, in a patient in need thereof, comprising the administration of a therapeutically effective amount of a PARG modulator.

### BACKGROUND

Poly(ADP-ribose) (PAR) oligomer or polymer chains are polymerized via Poly(ADP-ribose)ylation (PARylation) by PAR polymerase 1 (PARP1) and PAR chains are hydrolyzed via PARylation removal by poly(ADP-ribose) glycohydrolase (PARG), each of which critically regulate DNA damage responses. See Houl et al., Selective small molecule PARG inhibitor causes replication fork stalling and cancer cell death. Nat Commun. 2019 Dec. 11; 10 (1): 5654. PARG expression has been found to be upregulated in many cancers; and, the use of PARG inhibitors have been shown to sensitize cells to radiation-induced DNA damage; suppress replication fork progression; and impede cancer cell survival. Id.

PARG is a family of proteins expressed by a single gene alternatively spliced to generate five isoforms; PARG is an endo- and exo-glycohydrolase that rapidly degrades PAR generated by PARP proteins to coordinate DNA repair. See Gagne et al., The expanding role of poly(ADP-ribose) metabolism: current challenges and new perspectives. Curr. Opin. Cell Biol. 18, 145-151 (2006); and Min et al., Poly(ADP-ribose) glycohydrolase (PARG) and its therapeutic potential. Front. Biosci. (Landmark Ed.) 14, 1619-1626 (2009).

Breast cancer is the most commonly diagnosed cancer in women (with over 250,000 new cases estimated each year) and the second leading cause of cancer related death in women. Recent publications by The Cancer Genome Atlas (TCGA) Network and others have revealed fundamental molecular characteristics of breast cancer. Most notably, the four major breast cancer subtypes (luminal A, luminal B, human epidermal growth factor receptor 2 (HER2)-enriched, and basal-like) were identified and comprehensively analyzed revealing that breast cancers display a wide range of tumor heterogeneity caused by alterations in multiple factors.

Thus, there exists a need in the art for methods and compounds that can treat breast cancer. Disclosed herein, inter alia, are solutions to these and other problems in the art.

### SUMMARY

The present disclosure provides a method of treating breast cancer in a patient in need thereof, the method comprising: (a) selecting a patient having a breast cancer tumor wherein the tumor comprises homologous recombination deficiency (HRD) and is estrogen receptor (ER) positive (ER+); and (b) administering to the patient in need thereof, a therapeutically effective amount of a Poly(ADP-ribose) glycohydrolase (PARG) modulator, or a pharmaceutically acceptable salt thereof.

In addition, the present disclosure provides a method of treating breast cancer in a patient in need thereof, the method comprising: (a) selecting a patient having a breast cancer tumor wherein the tumor comprises homologous recombination deficiency (HRD) and is estrogen receptor (ER) positive (ER+); and (b) administering to the patient in need thereof, a therapeutically effective amount of a PARG modulator, or a pharmaceutically acceptable salt thereof; wherein the PARG modulator is a PARG inhibitor, for example, a compound of Formula (I) or a pharmaceutically acceptable salt thereof.

Also provided herein is a method of treating a homologous recombination deficiency (HRD) and estrogen receptor (ER) positive (ER+) breast cancer tumor in a patient in need thereof, the method comprising administering to the patient in need thereof, a therapeutically effective amount of a Poly(ADP-ribose) glycohydrolase (PARG) modulator, or a pharmaceutically acceptable salt thereof. In some embodiments, the PARG modulator is a PARG inhibitor, for example, a compound of Formula (I) or a pharmaceutically acceptable salt thereof.

Also provided herein is a Poly(ADP-ribose) glycohydrolase (PARG) modulator for use in a method of treating breast cancer in a patient in need thereof, wherein the patient has a breast cancer tumor and the tumor comprises homologous recombination deficiency (HRD) and is estrogen receptor (ER) positive (ER+), the method comprising administering a therapeutically effective amount of the PARG modulator to the patient in need thereof. In some embodiments, the PARG modulator is a PARG inhibitor, for example, a compound of Formula (I) or a pharmaceutically acceptable salt thereof.

Also provided herein is a Poly(ADP-ribose) glycohydrolase (PARG) modulator for use in a method of treating a homologous recombination deficiency (HRD) and estrogen receptor (ER) positive (ER+) breast cancer tumor, the method comprising administering a therapeutically effective amount of the PARG modulator to the patient in need thereof. In some embodiments, the PARG modulator is a PARG inhibitor, for example, a compound of Formula (I) or a pharmaceutically acceptable salt thereof.

Also provided herein is a a Poly(ADP-ribose) glycohydrolase (PARG) modulator for use in a method of treating breast cancer, said method comprising: (a) selecting a patient having a breast cancer tumor wherein the tumor comprises homologous recombination deficiency (HRD) and is estrogen receptor (ER) positive (ER+); and (b) administering to the patient in need thereof, a therapeutically effective amount of the Poly(ADP-ribose) glycohydrolase (PARG) modulator. In some embodiments, the PARG modulator is a PARG inhibitor, for example, a compound of Formula (I) or a pharmaceutically acceptable salt thereof.

Also provided herein is the use of a Poly(ADP-ribose) glycohydrolase (PARG) modulator in the manufacture of a medicament for treating breast cancer in a patient in need thereof, wherein the breast cancer in the patient is a tumor and the tumor comprises homologous recombination deficiency (HRD) and is estrogen receptor (ER) positive (ER+). In some embodiments, the PARG modulator is a PARG inhibitor, for example, a compound of Formula (I) or a pharmaceutically acceptable salt thereof.

Also provided herein is the use of a Poly(ADP-ribose) glycohydrolase (PARG) modulator in the manufacture of a medicament for treating a homologous recombination deficiency (HRD) and estrogen receptor (ER) positive (ER+) breast cancer tumor. In some embodiments, the PARG modulator is a PARG inhibitor, for example, a compound of Formula (I) or a pharmaceutically acceptable salt thereof.

Optionally, the breast cancer tumor in the methods, uses, and medicaments described herein is also progesterone receptor (PR or PgR) positive. Optionally, the breast cancer tumor in the methods, uses, and medicaments described herein is HER2−.

In addition, the present disclosure provides a method of treating cancer in a patient in need thereof, the method comprising administering to said patient a therapeutically effective amount of a (PARG) modulator, or a pharmaceutically acceptable salt thereof; wherein the PARG modulator is a PARG inhibitor, for example, a compound of Formula (I) or a pharmaceutically acceptable salt thereof.

### BRIEF DESCRIPTION OF THE DRAWINGS

FIG. 1: Shows cellular antiproliferative response to Compound A stratified by HR mutant status.

FIG. 2: Shows cell line derived xenograft (CDX) study with HCC1428 tumors using Compound B.

FIG. 3: Shows cell line derived xenograft (CDX) study with HCC1428 tumors using Compound A.

FIG. 4: Shows Patient-derived xenograft (PDX) study with HBCx-22 (Compound B).

FIG. 5: Shows Patient-derived xenograft (PDX) study with HBCx-22 (Compound A).

FIG. 6: Shows Patient-derived xenograft (PDX) study with HBCx-34 (Compound B).

FIG. 7: Shows Patient-derived xenograft (PDX) study with HBCx-34 (Compound A).

### DETAILED DESCRIPTION

### Definitions

When needed, any definition herein may be used in combination with any other definition to describe a composite structural group. By convention, the trailing element of any such definition is that which attaches to the parent moiety. For example, the composite group alkoxyalkyl means that an alkoxy group is attached to the parent molecule through an alkyl group.

“Administering” or “administration” or “administer” means to dispense, provide, and/or apply, and refers to any route of administration. For example, administering can refer to oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration can be by any route, including parenteral and transmucosal (e.g., buccal, sublingual, palatal, gingival, nasal, vaginal, rectal, or transdermal). In some embodiments, parenteral administration includes, e.g., intravenous, intramuscular, intra-arteriole, intradermal, subcutaneous, intraperitoneal, intraventricular, and intracranial. By “co-administer” it is meant that a compound or composition described herein is administered at the same time, just prior to, or just after the administration of one or more additional agents or therapies, also referred to herein as a “additional agent” (e.g. anti-cancer agent). The compound of the disclosure can be administered alone or can be co-administered to the patient. Co-administration is meant to include simultaneous or sequential administration of the compound individually or in combination (more than one compound or agent).

“Alkoxy,” and “haloalkoxy” refer to alkyl and haloalkyl groups respectively, each as defined herein, that is attached to the remainder of the molecule via an oxygen atom.

“Alkyl” by itself or as part of another substituent, means, unless otherwise stated, a saturated straight or branched chain hydrocarbon radical, having the number of carbon atoms designated (i.e. C₁₋₈ means one to eight carbons). Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. Examples of alkyl groups include methyl, ethyl, n-propyl, isopropyl, n-butyl, t-butyl, isobutyl, sec-butyl, n-pentyl, n-hexyl, n-heptyl, n-octyl, and the like.

“Ameliorate” or “amelioration” includes the arrest, prevention, decrease, or improvement in one or more the symptoms, signs, and features of the disease being treated, both temporary and long-term.

“Aryl” means, unless otherwise stated, an aromatic, hydrocarbon group which can be a single ring or multiple rings (up to three rings) which are fused together or linked covalently. Non-limiting examples of aryl groups include phenyl, naphthyl and biphenyl.

“Biological sample” refers to any specimen, e.g., such as cells, tissue (e.g., a tissue sample obtained by biopsy), blood, serum, plasma, urine, cerebrospinal fluid, and/or in pancreatic fluid, taken from a subject. Preferably, the sample is taken from a portion of the body affected by a cancer (e.g., a biopsy of the cancer tissue, such as breast cancer tissue). Biopsy may involve fine needle aspiration biopsy, core needle biopsy (e.g., stereotactic core needle biopsy, vacuum-assisted core biopsy, or magnetic resonance imaging (MRI) guided biopsy), or surgical biopsy (e.g., incisional biopsy or excisional biopsy). The sample may undergo additional purification and processing, for example, to remove cell debris and other unwanted molecules. Additional processing may further involve producing cDNA molecules corresponding to nucleic acid molecules (e.g., mRNA) in the sample and/or amplification of the nucleic acid molecules, e.g., using PCR, such as RT-PCR. The standard methods of sample purification, such as removal of unwanted molecules, are known in the art.

“Biomarker” refers to an indicator, e.g., a predictive, diagnostic, and/or prognostic indicator, which can be detected in a sample. The biomarker may serve as an indicator of a particular subtype of a disease or disorder (e.g., cancer) characterized by certain, molecular, pathological, histological, and/or clinical features. In some embodiments, the biomarker is a gene. In some embodiments, the biomarker is a variation (e.g., mutation and/or polymorphism) of a gene. In some embodiments, the biomarker is a translocation. Biomarkers include, but are not limited to, polynucleotides (e.g., DNA, and/or RNA), polypeptides, polypeptide and polynucleotide modifications (e.g., posttranslational modifications), carbohydrates, and/or glycolipid-based molecular markers. In some embodiments, a biomarker may be HRD; ER; PR; or HER2.

“Breast cancer,” refers to any clinical diagnosis of breast cancer, and includes any and all particular sub-phenotypes of breast cancer. For example, breast cancer is sometimes categorized as estrogen receptor (ER) positive (ER+); breast cancer is sometimes also categorized as progesterone receptor (PR) positive (PR+). Breast cancer is furthermore sometimes diagnosed as invasive ductal, as invasive lobular, as tubular, or as otherwise invasive or mixed invasive. Breast cancer can also be categorized as medullary DCIS (Ductal Carcinoma In-Situ) or LCIS (Lobular Carcinoma In-Situ, or otherwise non-invasive. Invasive breast cancer can also be defined as stage 0, stage 1, stage 2 (including stage 2a and stage 2b), stage 3 (including stage 3a, stage 3b and stage 3c) or stage 4 breast cancer. In the present context, “breast cancer” can include any of these sub-phenotypes of breast cancer, and also includes any other clinically applicable sub-phenotypes of breast cancer.

“cDNA” or “copy DNA” or “complementary DNA” refers to a molecule that is complementary to a molecule of RNA. In some embodiments, cDNA may be either single-stranded or double-stranded. In some embodiments, cDNA can be a double-stranded DNA synthesized from a single stranded RNA template in a reaction catalyzed by a reverse transcriptase. In yet other embodiments, “cDNA” refers to all nucleic acids that share the arrangement of sequence elements found in native mature mRNA species, where sequence elements are exons and 3′ and 5′ non-coding regions. Normally mRNA species have contiguous exons, with the intervening introns removed by nuclear RNA splicing, to create a continuous open reading frame encoding the protein. In some embodiments, “cDNA” refers to a DNA that is complementary to and derived from an mRNA template.

“Combination” refers to simultaneous, separate, or sequential administration. For example, in some embodiments, “combination” refers to simultaneous administration. In another embodiment, “combination” refers to separate administration. In a further embodiments, of the disclosure “combination” refers to sequential administration. Where the administration is sequential or separate, the delay in administering the second component should not be such as to lose the beneficial effect of the combination.

“DNA” refers to deoxyribonucleic acid, comprising a polymer of one or more deoxyribonucleotides or nucleotides (i.e., adenine [A], guanine [G], thymine [T], or cytosine [C]), which can be arranged in single-stranded or double-stranded form. For example, one or more nucleotides creates a polynucleotide.

“Estrogen receptor” or “ER” refers to an established member of the nuclear receptor family of receptors which is a transcription factor activated by binding ligands such as the hormones 17β-estradiol, estriol, estrone, and the like. The term “estrogen receptor” may refer to the nucleotide sequence or protein sequence of human estrogen receptor. The term “estrogen receptor” may refer to the nucleotide sequence or protein sequence of human estrogen receptor 1 (a.k.a. ER-alpha, ERalpha, or ERα) (e.g., Entrez 2099, Uniprot P03372, RefSeq NM_000125, OMIM 133430, NP_000116, NP_000116.2, NM_000125.3, GI: 62821794, and/or GI: 170295798). The term “estrogen receptor” may refer to the nucleotide sequence or protein sequence of human estrogen receptor 2 (a.k.a. ER-beta, ERbeta, or ERB) (e.g., Entrez 2100, Uniprot Q92731, RefSeq NM_001040275, OMIM 601663, and/or GI: 94538324). The term “estrogen receptor” includes both the wild-type form of the nucleotide sequences or proteins as well as any mutants thereof. In some embodiments, “estrogen receptor” is wild-type estrogen receptor. In some embodiments, “estrogen receptor” is one or more mutant forms. In some embodiments, an estrogen receptor is the wildtype human estrogen receptor. In embodiments, an estrogen receptor is the wildtype human ERα. In some embodiments, an estrogen receptor is the wildtype human ERB. In some embodiments, an estrogen receptor is the wildtype human ERα or ERβ. In some embodiments, an estrogen receptor is the wildtype human ERα and ERβ. In embodiments, the estrogen receptor is a mutant estrogen receptor. In embodiments, the mutant estrogen receptor is associated with a disease that is not associated with wildtype estrogen receptor (e.g., drug resistant cancer). In some embodiments, the estrogen receptor includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations).

“Estrogen receptor positive breast cancer” or “ER-positive breast cancer” or “ER+ breast cancer” refers to tumors determined to be positive for estrogen receptor. In the present context, ER levels of greater than or equal to 10 fmol/mg and/or an immunohistochemical observation of greater than or equal to 10% positive nuclei is considered to be ER positive. Breast cancer that does not fulfill the criteria of being ER positive is defined herein as “ER negative” or “ER-” or “estrogen receptor negative.”

“Halo” or “halogen,” by itself or as part of another substituent, mean, unless otherwise stated, a fluorine, chlorine, bromine, or iodine atom.

“Haloalkyl,” means alkyl, as defined above, that is substituted with one to five halo atoms and includes monohaloalkyl and polyhaloalkyl. For example, the term “C₁₋₄ haloalkyl” includes trifluoromethyl, 2,2,2-trifluoroethyl, 4-chlorobutyl, 3-bromopropyl, and the like.

“HER2” and “ErbB2” and “HER2 receptor” and “HER-2/neu” and “Her2” as used interchangeably herein, refer to the protein product of the human neu oncogene, also referred to as the ErbB2 oncogene or the HER2 oncogene.

“Heteroaryl” refers to a 5- to 10-membered aromatic ring that contains from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like.

“Heteroatom” is meant to include oxygen (O), nitrogen (N), sulfur(S) and silicon (Si).

“Heterocycloalkyl” or “heterocyclyl” refers to a saturated or partially unsaturated 4 to 10 membered monocyclic or bicyclic ring having from one to four heteroatoms independently selected from N, O, and S and the remaining ring atom being carbon. The nitrogen and sulfur atoms are optionally oxidized, and the nitrogen atom(s) are optionally quaternized and one or two ring carbon atoms of the heterocyclic ring may be replaced by —C═(O) group. Non limiting examples of heterocycloalkyl groups include pyrrolidine, imidazolidine, pyrazolidine, butyrolactam, valerolactam, imidazolidinone, hydantoin, dioxolane, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, and the like. A heterocycloalkyl group can be attached to the remainder of the molecule through a ring carbon or a heteroatom. Non limiting examples of heterocycloalkyl groups include pyridine-2 (H)-one.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. Thus, in some embodiments, the term “homologous” refers to the sequence similarity between two polypeptide molecules, or between two nucleic acid molecules. When a position in both of the two compared sequences is occupied by the same base or amino acid monomeric subunit, e.g., if a position in each of two DNA molecules is occupied by adenine, then the molecules are homologous at that position. The homology between two sequences is a function of the number of matching or homologous positions shared by the two sequences. For example, if 6 of 10 of the positions in two sequences are matched or homologous then the two sequences are 60% homologous. By way of example, the DNA sequences ATTGCC and TATGGC share 50% homology.

As used herein “homologous recombination” refers to the cellular process of genetic recombination in which nucleotide sequences are exchanged between two similar or identical DNA sequences.

The term “Homologous recombination deficiency” or “HRD” as used interchangeably herein, refer to a phenotype that is characterized by the inability of a cell to effectively repair DNA double-strand breaks using homologous recombination repair (HRR) pathway. HR deficiency may arise from absence or reduction of one or more HR-associated genes or presence of one or more mutations in one or more HR-associated genes. Examples of HR-associated genes include BRCA1, BRCA2, RAD54, RAD51B, ATM, BARD1, CHECK1, CHECK2, CDK12, RAD51B, RAD54L, RAD51D, PPP22A, BRIP1, CtIP (CtBP-interacting protein), PALB2 (Partner and Localizer of BRCA2), XRCC2 (X-ray repair complementing defective repair in Chinese hamster cells 2), RECQL4 (RecQ Protein-Like 4), BLM (Bloom syndrome, RecQ helicase-like), WRN (Werner syndrome, one or more HR-associated genes) Nbs 1 (Nibrin), and genes encoding Fanconi anemia (FA) proteins or FA-like genes e.g, FANCA, FANCB, FANCC, FANCD1 (BRCA2), FANCD2, FANCE, FANCF, FANCG, FANCI, FANJ (BRIP1), FANCL, FANCM, FANCN (RALB2), FANCP (SLX4), FANCS (BRCA1), RAD51C, and XPF. Exemplary methodology used to classify tumors as HRD include HRD score (unweighted sum of the scores for three ultrastructural features: large-scale state transitions (LST), telomere allelic imbalances (TAI), and loss-of-heterozygosity (LOH)), HRDetect, single base substitution signature 3 (SBS3), and indel signature 6 (ID6). See Moore G M, et al., Examining the prevalence of homologous recombination repair defects in ER+ breast cancers. Breast Cancer Res Treat. 2022 April; 192 (3): 649-653.

The term “homology,” when used in relation to nucleic acids, refers to a degree of complementarity. There may be partial homology, or complete homology and thus identical. “Sequence identity” refers to a measure of relatedness between two or more nucleic acids, and is given as a percentage with reference to the total comparison length. The identity calculation takes into account those nucleotide residues that are identical and in the same relative positions in their respective larger sequences.

“Hydroxyalkyl,” means alkyl, as defined above, that is substituted with one or two hydroxy. For example, the term “hydroxyC₁₋₄ alkyl” is mean to include hydroxymethyl, 1-, or 2-hydroxyethyl, 1,2-dihydroxyethyl, hydroxypropyl, and the like.

“IC₅₀” or the half maximal inhibitory concentration (IC₅₀) is a measure of the potency of a substance in inhibiting a specific biological or biochemical function. IC₅₀ is a quantitative measure that indicates how much of a particular inhibitory substance is needed to inhibit a given biological process or biological component by 50%.

“Inhibiting” and “reducing,” or any variation of these terms in relation of PARG, includes any measurable decrease or complete inhibition to achieve a desired result. For example, there may be a decrease of about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more, reduction of PARG activity compared to normal. About as used herein means within ±10%, preferably ±5% of a given value.

“Mutant” or “mutation” refers to an organism, DNA sequence, peptide sequence, or polypeptide sequence, that has an alteration (for example, in the DNA sequence), which causes said organism and/or sequence to be different from the normally, non-deleterious state of existence that typically occurs in healthy patients and/or sequences.

“NCBI” refers to the National Center for Biotechnology Information.

“PARG” refers to poly(ADP-ribose) glycohydrolase (PARG).

“PARG modulator” refers to one or more chemicals, organic compounds, inorganic compounds, small molecules, molecules, nucleotides, polynucleotides, RNA, DNA, peptides, polypeptides, proteins, lipids, glycolipids, enzymes, toxins, toxicants, poisons, insecticides, pesticides, viruses, prokaryote organisms (and/or agents produced therefrom), eukaryote organisms (and/or the agents produced therefrom), that modulates the expression and/or activity of PARG. In some embodiments, a PARG modulator may be organic or inorganic; small to large molecular weight individual compounds; mixtures and combinatorial libraries of inhibitors, agonists, or antagonists. In some embodiments, a PARG modulator may be a natural product or a naturally-occurring small molecule organic compound. In some embodiments, a PARG modulator may be a carbohydrate; monosaccharide; oligosaccharide; polysaccharide; amino acid; peptide; oligopeptide; polypeptide; protein; receptor; nucleic acid; nucleoside; nucleotide; oligonucleotide; polynucleotide including DNA and DNA fragments, RNA and RNA fragments and the like; lipid; retinoid; steroid; glycopeptides; glycoprotein; proteoglycan and the like; and synthetic analogues or derivatives thereof, including peptidomimetics, small molecule organic compounds and the like, and mixtures thereof. In some embodiments, a PARG modulator can inhibit PARG.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient” and “subject” are used herein interchangeably, and refer to any animal (e.g., a mammal, such as a human, a laboratory animal, such as a mouse, rat, rabbit, guinea pig, or other animal models of cancer, or a domesticated animal, such as a dog, cat, or a domesticated animal, for example, sheep, horses, cattle, pigs and goats). A patient in need of treatment, according to the methods described herein, may be one who has been diagnosed with a cancer, such as those described herein, including, without limitation, breast cancer, ovarian cancer, endometrial cancer, gastric, prostate, and/or colorectal cancer. In some embodiments, Patient is human.

“Pharmaceutically acceptable salts” is meant to include salts of the compounds of present disclosure, which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Non-limiting examples of salts derived from pharmaceutically acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds of the present disclosure may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

“Progesterone receptor positive breast cancer” or “PR-positive breast cancer” or “PR+ breast cancer” refers to tumors determined to be positive for progesterone receptor. In the present context, PR levels of greater than or equal to 10 fmol/mg and/or an immunohistochemical observation of greater than or equal to 10% positive nuclei is considered to be PR positive. Breast cancer that does not fulfill the criteria of being PR positive is defined herein as “PR negative” or “progesterone receptor negative.”

“Proliferative disorder” refers to an unwanted or uncontrolled cellular proliferation of excessive or abnormal cells that is undesired, such as, neoplastic or hyperplastic growth. Examples of proliferative conditions include, but are not limited to, pre-malignant and malignant cellular proliferation, including but not limited to, malignant neoplasms and tumors, cancers, leukemias, psoriasis, bone diseases, fibroproliferative disorders (e.g., of connective tissues), and atherosclerosis. Any type of cell may be treated, including but not limited to, lung, colon, breast, ovarian, prostate, liver, pancreas, brain, and skin.

“Protecting group” refers to a moiety or functionality that is introduced into a molecule by chemical modification of a functional group in order to obtain chemoselectivity in a subsequent chemical reaction. Standard protecting groups are provided in Wuts and Greene: “Greene's Protective Groups in Organic Synthesis,” 4th Ed, Wuts, P. G. M. and Greene, T. W., Wiley-Interscience, New York: 2006, the disclosures of which are incorporated herein by reference in their entireties.

“Small molecule” refers to any organic compound or salt thereof, whether naturally-occurring or artificially created (e.g., via chemical synthesis) that has a relatively low molecular weight (e.g., a molecular weight no greater than 6 kDa). In some embodiments, a small molecule can have a molecular weight (MW) less than or equal to about 5 kDa. In other embodiments, a small molecule can have a MW of less than or equal to about 3 kDa. In other embodiments, a small molecule can have a MW of less than or equal to about 1.5 kDa. In other embodiments, a small molecule can have a MW of less than or equal to about 1.0 kDa. In some embodiments, small molecules may include repeat units e.g., using a long chain alkyl group as a substituent does not remove a molecule from the “small molecule” class. In some embodiments, small molecules may be nucleic acids, peptides, polypeptides, peptidomimetics, carbohydrates, lipids or other organic (carbon-containing) or molecules.

“Substantially pure” indicates that a component makes up greater than about 50% of the total content of the composition, and typically greater than about 60% of the total content. More typically, “substantially pure” refers to compositions in which at least 75%, at least 85%, at least 90% or more of the total composition is the component of interest.

“Therapeutically effective amount” or “effective amount” or “pharmaceutically effective amount” refer to a nontoxic but sufficient amount of one or more compounds described herein, or a pharmaceutically acceptable salt thereof, to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation. By way of example, measurement of the serum level of a PARG inhibitor (or, e.g., a metabolite thereof) at a particular time post-administration may be indicative of whether a therapeutically effective amount has been used.

“Treatment” or “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. In some embodiments, “treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently.

Throughout this specification, unless the context requires otherwise, the word “comprise,” or variations such as “comprises” or “comprising,” will be understood to imply the inclusion of a stated step or element or integer or group of steps or elements or integers but not the exclusion of any other step or element or integer or group of elements or integers.

All patent applications, patents, and printed publications referred to herein are incorporated by reference in their entirety to the same extent as if each individual publication, patent, or patent application was specifically and individually indicated to be incorporated by reference in its entirety. And, all patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

### Compounds of the Present Disclosure

In some embodiments, a compound of the present disclosure is a PARG modulator. In some embodiments, the PARG modulator is a PARG inhibitor. In some embodiments, the PARG modulator is a small molecule PARG inhibitor.

In some embodiments, a compound of the present disclosure is a PARG modulator such as a compound of Formula (I) or a pharmaceutically acceptable salt thereof. The compounds of the present disclosure are described in detail below.

### Formula (I)

In some embodiments, a PARG modulator is a compound of Formula (I):

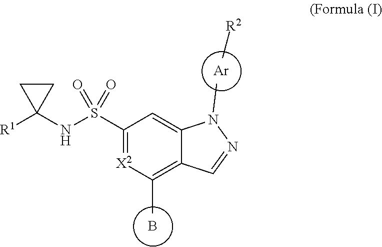

or a pharmaceutically acceptable salt thereof, wherein
R¹ is selected from the group consisting of cyano, C₁₋₂ alkyl, and C₁₋₂ haloalkyl;
Ar is a 5-membered heteroaryl;
X² is CH or CF;
R² is selected from the group consisting of C₁₋₃ alkyl, C₁₋₃ haloalkyl, hydroxyC₁₋₃alkyl, and cyano;
ring B is 5- or 6-membered heterocycloalkyl substituted with R^{a}, R^{b}, and R^{c};
R^{a} is hydrogen, C₁₋₄ alkyl, C₁₋₄ haloalkyl, halo, hydroxy, —C(O)R^{d} (where R^{d} is hydrogen, C₁₋₆ alkyl, or C₁₋₆ haloalkyl); and
R^{b} and R^{c} are independently selected from hydrogen, C₁₋₆ alkyl, hydroxy, C₁₋₆ alkoxy, halo, C₁₋₆ haloalkyl, and C₁₋₆ haloalkoxy.

In some embodiments of Formula (I) or a pharmaceutically acceptable salt thereof, X² is CH. In some embodiments of Formula (I) or a pharmaceutically acceptable salt thereof, X² is CF.

In some embodiments of Formula (I) or a pharmaceutically acceptable salt thereof, R¹ is cyano. In some embodiments of Formula (I) or a pharmaceutically acceptable salt thereof, R¹ is C₁₋₂ alkyl. In some embodiments of Formula (I) or a pharmaceutically acceptable salt thereof, R¹ is methyl or ethyl. In some embodiments of Formula (I) or a pharmaceutically acceptable salt thereof, R¹ is methyl.

In some embodiments of Formula (I) or a pharmaceutically acceptable salt thereof, Ar is 1,2,4-thiadiazolyl, or 1,3,4-thiadiazolyl. In some embodiments of Formula (I) or a pharmaceutically acceptable salt thereof, Ar is 1,2,4-thiadiazolyl. In some embodiments of Formula (I), Ar is 1,3,4-thiadiazolyl.

In some embodiments of Formula (I) or a pharmaceutically acceptable salt thereof, R² is attached to the carbon atom of Ar that is meta to the atom of Ar that is attached to the nitrogen atom of the remainder of the molecule.

In some embodiments of Formula (I) or a pharmaceutically acceptable salt thereof, R² is methyl, ethyl, difluoromethyl, trifluoromethyl, cyano. In some embodiments of Formula (I) or a pharmaceutically acceptable salt thereof, R² is difluoromethyl.

In some embodiments of Formula (I) or a pharmaceutically acceptable salt thereof, ring B is 5-membered heterocycloalkyl. In some embodiments of Formula (I) or a pharmaceutically acceptable salt thereof, ring B is 6-membered heterocycloalkyl. In some embodiments of Formula (I) or a pharmaceutically acceptable salt thereof, ring B is morpholinyl, 1,1-dioxothiomorpholinyl, pyrrolidinyl, piperidinyl, 6-oxo-1,6-dihydropyridinyl, or piperazinyl. In some embodiments of Formula (I) or a pharmaceutically acceptable salt thereof, ring B is morpholinyl, pyrrolidinyl, piperidinyl, or piperazinyl. In some embodiments of Formula (I) or a pharmaceutically acceptable salt thereof, ring B is piperazinyl.

In some embodiments of Formula (I) or a pharmaceutically acceptable salt thereof, R^{a} is hydrogen, C₁₋₄ alkyl, C₁₋₄ haloalkyl, halo, hydroxy, —C(O)R^{d} (where R^{d} is hydrogen, C₁₋₆ alkyl, or C₁₋₆ haloalkyl); and R^{b} and R^{c} are independently selected from hydrogen, C₁₋₆ alkyl, hydroxy, C₁₋₆ alkoxy, halo, C₁₋₆ haloalkyl, and C₁₋₆ haloalkoxy.

In some embodiments of Formula (I) or a pharmaceutically acceptable salt thereof, R^{a} is hydrogen, C₁₋₄ alkyl, C₁₋₄ haloalkyl, —C(O)R^{d} (where R^{d} is C₁₋₆ alkyl, or C₁₋₆ haloalkyl); and R^{b} and R^{c} are independently selected from hydrogen, C₁₋₆ alkyl, and C₁₋₆ haloalkoxy.

In some embodiments of Formula (I) or a pharmaceutically acceptable salt thereof, R^{a} is —C(O)R^{d} where R^{d} is C₁₋₆ alkyl; and R^{b} and R^{c} are independently selected from hydrogen, C₁₋₆ alkyl, and C₁₋₆ haloalkoxy.

In some embodiments of Formula (I) or a pharmaceutically acceptable salt thereof, R^{a} is hydrogen C₁₋₄ alkyl, and C₁₋₄ haloalkyl; and R^{b} and R^{c} are independently selected from hydrogen, and C₁₋₆ alkyl.

In an embodiment of Formula (I) or a pharmaceutically acceptable salt thereof, the compound is:

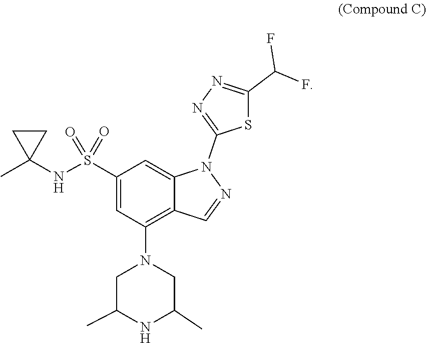 [SMILES: CC1CN(c2cc(S(=O)(=O)NC3(C)CC3)cc3c2cnn3-c2nnc(C(F)F)s2)CC(C)N1]

In an embodiment of Formula (I), the compound is Compound C.

In an embodiment of Formula (I) or a pharmaceutically acceptable salt thereof, the compound is selected from the group consisting of:

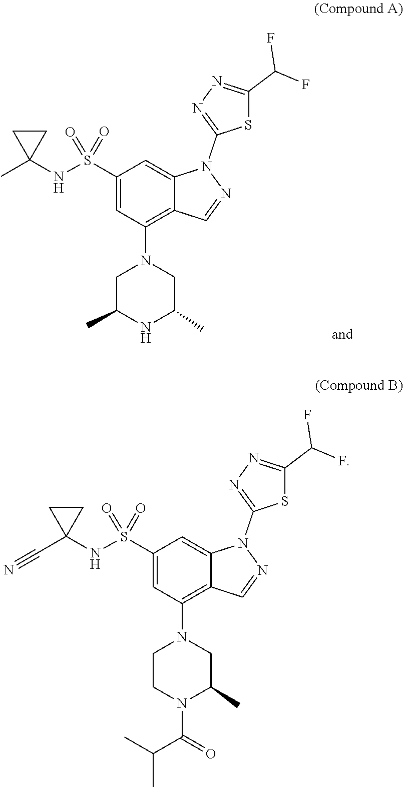 [SMILES: C[C@H]1CN(c2cc(S(=O)(=O)NC3(C)CC3)cc3c2cnn3-c2nnc(C(F)F)s2)C[C@H](C)N1 ; CC(C)C(=O)N1CCN(c2cc(S(=O)(=O)NC3(C#N)CC3)cc3c2cnn3-c2nnc(C(F)F)s2)C[C@H]1C]

In an embodiment of Formula (I) or a pharmaceutically acceptable salt thereof, the compound is:

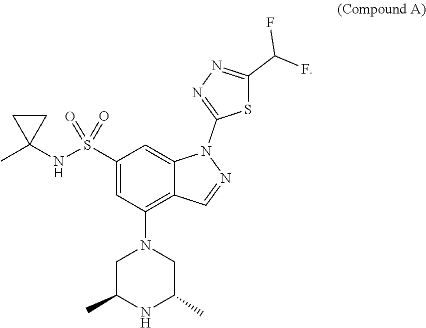 [SMILES: C[C@H]1CN(c2cc(S(=O)(=O)NC3(C)CC3)cc3c2cnn3-c2nnc(C(F)F)s2)C[C@H](C)N1]

In an embodiment of Formula (I), the compound is Compound A.

In an embodiment of Formula (I) or a pharmaceutically acceptable salt thereof, the compound is:

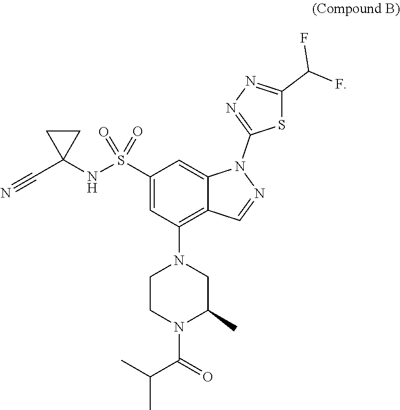 [SMILES: CC(C)C(=O)N1CCN(c2cc(S(=O)(=O)NC3(C#N)CC3)cc3c2cnn3-c2nnc(C(F)F)s2)C[C@H]1C]

In an embodiment of Formula (I), the compound is Compound B.

In an embodiment of Formula (I) or a pharmaceutically acceptable salt thereof, the compound is

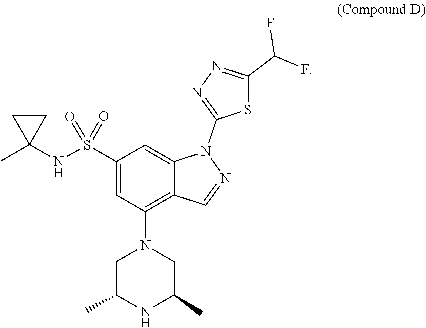 [SMILES: C[C@@H]1CN(c2cc(S(=O)(=O)NC3(C)CC3)cc3c2cnn3-c2nnc(C(F)F)s2)C[C@@H](C)N1]

In an embodiment of Formula (I), the compound is Compound D.

In an embodiment of Formula (I) or a pharmaceutically acceptable salt thereof, the compound is

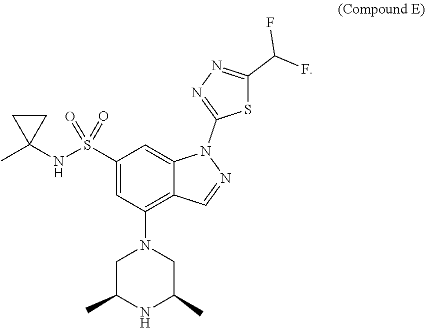 [SMILES: C[C@@H]1CN(c2cc(S(=O)(=O)NC3(C)CC3)cc3c2cnn3-c2nnc(C(F)F)s2)C[C@H](C)N1]

In an embodiment of Formula (I), the compound is Compound E.

In an embodiment of Formula (I) or a pharmaceutically acceptable salt thereof, the compound is selected from the group consisting of Compound A, Compound B, Compound D, and Compound E.

In an embodiment of Formula (I), the compound is selected from the group consisting of Compound A, Compound B, Compound D, and Compound E.

Any of the foregoing compounds of Formula (I) or a pharmaceutically acceptable salt thereof can be used to practice the methods of the present disclosure. For example, any of the foregoing compounds of Formula (I), or a pharmaceutically acceptable salt thereof or a composition comprising the same, can be used in the present disclosure's method to treat breast cancer in a patient in need thereof, the method comprising selecting a patient having a breast cancer tumor wherein the tumor comprises homologous recombination deficiency (HRD) and is estrogen receptor (ER) positive (ER+), and administering to the patient a therapeutically effective amount of a PARG modulator.

### How to Make Compounds of the Present Disclosure

The compounds of the present disclosure can be prepared by any suitable technique known in the art. Particular processes for the preparation of these compounds are described further in the accompanying examples.

Exemplary descriptions regarding preparation of the compounds of the present disclosure are provided in WO2021/055744, the disclosure of which is incorporated herein by reference in its entirety.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

Those having ordinary skill in the art will recognize that the functionality present on various portions of the molecule must be compatible with the reagents and reaction conditions utilized.

In some embodiments, during the synthesis of the compounds of the disclosure in the processes defined herein, or during the synthesis of certain starting materials, it may be desirable to protect certain substituent groups to prevent their undesired reaction. The skilled artisan will appreciate when such protection is required, and how such protecting groups may be put in place, and later removed.

Exemplary descriptions of protecting groups and methods concerning the same are provided in: “Protective Groups in Organic Synthesis” by Theodora Green (publisher: John Wiley & Sons), the disclosure of which is incorporated herein by reference in its entirety.

In some embodiments, protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

In some embodiments, once a compound of the present disclosure has been synthesized by any one of the processes described herein, the processes may then further comprise the additional steps of: (i) removing any protecting groups present; (ii) converting the compound into another compound (e.g., with another group); and (iii) optionally forming a pharmaceutically acceptable salt thereof.

In some embodiments, a compound of the present disclosure can be isolated and purified using any technique well known in the art.

Exemplary descriptions of how to make the compounds of the present disclosure are provided in WO2021/055744; Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999); the disclosures of which are incorporated herein by reference in their entireties.

### Methods of Use and Administration

The present disclosure provides compounds that function as modulators of PARG, for use in a method for the treatment and/or prevention of certain breast cancer tumor in a patient in need thereof, wherein the tumor comprises homologous recombination deficiency (HRD) and are ER positive, (and optionally progesterone receptor (PR or PgR) positive, and/or optionally a HER2-tumor); and administering to the patient a therapeutically effective amount of a PARG modulator.

In some embodiments, the present disclosure provides a method comprising administering to the subject a therapeutically effective amount of one or more compounds described herein, or a pharmaceutically acceptable salt thereof, which is optionally in a composition. In certain embodiments, the method further comprises administering to the subject an additional therapeutic agent that treats or prevents cancer.

In some embodiments, the present disclosure provides a method comprising contacting a subject or a cell therefrom with an effective amount of a compound, or a pharmaceutically acceptable salt thereof, as defined herein.

In some embodiments, the present disclosure provides a method comprising treating a disease or disorder in which PARG activity is implicated in a subject in need of such treatment, said method comprising administering to said patient a therapeutically effective amount of a compound, or a pharmaceutically acceptable salt thereof, or a pharmaceutical composition as defined herein.

In some embodiments, the present disclosure provides a method comprising treating a proliferative disorder in a patient in need of such treatment, said method comprising administering to said patient a therapeutically effective amount of a compound, or a pharmaceutically acceptable salt thereof, or a pharmaceutical composition as defined herein.

In some embodiments, the present disclosure provides a method comprising treating cancer in a subject in need of such treatment, said method comprising administering to said subject a therapeutically effective amount of a compound, or a pharmaceutically acceptable salt thereof, or a pharmaceutical composition as defined herein.

In some embodiments, the present disclosure comprises the use of one or more of the compounds described herein for therapeutic purposes.

In some embodiments, the present disclosure comprises the use of one or more of the compounds described herein for prophylactic purposes.

Any of the compounds of the present disclosure, i.e., compounds of Formula (I) or a pharmaceutically acceptable salt thereof, or a composition comprising the same; can be used to practice the methods of the present disclosure. For example, any of the compounds of Formula (I), or a pharmaceutically acceptable salt thereof, or composition comprising the same; can be used in the method contemplated by the present disclosure to treat breast cancer in a patient in need thereof, wherein the method comprises: selecting a patient having a breast cancer tumor wherein the tumor comprises homologous recombination deficiency (HRD) and is estrogen receptor (ER) positive; and administering to the patient a therapeutically effective amount of a PARG modulator. As a further example, any of the compounds of Formula (I), or a pharmaceutically acceptable salt thereof, or composition comprising the same; can be used in the method contemplated by the present disclosure to treat a homologous recombination deficiency (HRD) and estrogen receptor (ER) positive (ER+) breast cancer in a patient in need thereof, wherein the method comprises administering to the patient a therapeutically effective amount of a PARG modulator.

As another example, any of the compounds of the present disclosure, i.e., Formula (I), or a pharmaceutically acceptable salt thereof, or composition comprising the same; can be used in a method to treat a cancer in a patient in need thereof, wherein the method comprises administering to the patient a therapeutically effective amount of a PARG modulator. In an embodiment, the cancer is ovarian cancer, prostate cancer, colorectal cancer, gastric cancer, or endometrial cancer. In an embodiment, the cancer is ovarian cancer, prostate cancer, pancreatic cancer, colorectal cancer, gastric cancer, or endometrial cancer. In an embodiment, provided is a method of treating a cancer in a patient in need thereof, the method comprising administering to said patient a therapeutically effective amount of a (PARG) modulator or a composition comprising the PARG modulator; wherein the PARG modulator is a PARG inhibitor, for example, a compound of Formula (I) or a pharmaceutically acceptable salt thereof. In an embodiment, the cancer is ovarian cancer, prostate cancer, pancreatic cancer, colon cancer, rectal cancer, gastric cancer, skin cancer, endometrial cancer, cervical cancer, brain cancer, liver cancer, bladder cancer, esophageal cancer, kidney cancer, rectal cancer, stomach cancer, thyroid cancer, lymphoma, leukemia, melanoma, uterine cancer, nabtle cell lymphoma, renal cell carcinoma, appendicle cancer, hematologic cancer, MYH-related polyposis, gallbladder cancer, bile duct cancer, testicular cancer, bone cancer, or head and neck cancer. In an embodiment, the cancer is skin cancer or head and neck cancer. PARG modulators disclosed herein can be used to treat cancers exhibiting or displaying DNA replication stress. For example, DNA replication stress can be due to defective DNA damage repair mechanism.

### Biomarkers

In some embodiments, a biomarker can be a protein selected from the set of proteins provided by the present disclosure and whose expression profile was found to be indicative of cancer (e.g., breast cancer). In other embodiments, a biomarker can be a polynucleotide or nucleic acid molecule comprising a nucleotide sequence, which codes for a biomarker protein of the present disclosure, as well as polynucleotides that hybridize with portions of these nucleic acid molecules.

In some embodiments, a biomarker may be indicative of cancer when said biomarker possesses an expression pattern or profile, which is diagnostic of cancer, such that the expression pattern is found significantly more often in subjects with cancer than in patients without cancer.

In some embodiments, a biomarker may be differentially expressed in a subject suffering from a disease state or condition. For example, in some embodiments, a biomarker's abundance level is different in a subject (or a population of subjects) afflicted with a disease or condition relative to the biomarker's level in a healthy or normal subject (or a population of healthy or normal subjects). Differential expression or level of the biomarker includes quantitative, as well as qualitative, differences in the temporal or cellular expression pattern of the biomarker.

In some embodiments, a differentially expressed biomarker, alone or in combination with other differentially expressed biomarkers, is useful in a variety of different applications in diagnostic, sub-typing, therapeutic, drug development and related areas. The expression patterns and/or levels of one or more differentially expressed biomarkers can be described as a fingerprint or a signature of a disease state or condition, disease subtype, and/or stage in the disease state's progression.

In other embodiments, the differential levels of one or more biomarkers can be used as a point of reference to compare and characterize unknown samples and samples for which further information is sought.

In some embodiments, the term “decreased level” as it pertains to the level of biomarker, can refer to a decrease in the abundance level of one or more biomarkers of at least 1%, 2%, 3%, 4%, 5%, 6%, 7%, 8%, 9%, 10%, 15%, 20%, 25%, 30%, 40%, 50%, 60%, 70%, 80%, 90%, 100%, or more, or a decrease of greater than 1-fold, 2-fold, 3-fold, 4-fold, 5-fold, 10-fold, 50-fold, 100-fold or more as measured by one or more methods described herein.

In some embodiments, the term “increased level” as it pertains to the level of biomarker, can refer to an increase in the abundance one or more biomarkers of at least 1%, 2%, 3%, 4%, 5%, 6%, 7%, 8%, 9%, 10%, 15%, 20%, 25%, 30%, 40%, 50%, 60%, 70%, 80%, 90%, 100%, or more, or an increase of greater than 1-fold, 2-fold, 3-fold, 4-fold, 5-fold, 10-fold, 50-fold, 100-fold or more as measured by one or more methods, such as method described herein.

In some embodiments, the biomarker may serve as an indicator of a particular subtype of a disease or disorder (e.g., a cancer, such as breast cancer) characterized by certain, molecular, pathological, histological, and/or clinical features.

In some embodiments, the biomarker is a gene. In some embodiments, the biomarker is a variation (e.g., mutation and/or polymorphism) of a gene. In some embodiments, the biomarker is a translocation.

In some embodiments, biomarkers include, but are not limited to, polynucleotides (e.g., DNA, and/or RNA), polypeptides, polypeptide and polynucleotide modifications (e.g., posttranslational modifications), carbohydrates, and/or glycolipid-based molecular markers.

In some embodiments, a biomarker may be HRD; ER; PR; or HER2. In other embodiments, a biomarker can be a gene and/or protein that is transcribed and/or translated, respectively, in response to the activity of HRD, ER, PR, and/or HER2. For example, in some embodiments, a biomarker can be upstream or downstream of HRD, ER, PR and HER2 in their respective signaling pathways.

In some embodiments, a biomarker can be a gene or protein associated with a cancer subtype, and/or progression of disease. In some embodiments, overexpression of the biomarker or combination of biomarkers may be indicative of either a good prognosis (i.e., disease-free survival) or a bad prognosis (i.e., cancer recurrence, metastasis, or death from the underlying cancer). Thus, in some embodiments, present method an assessment of a biomarker (e.g., in a biological sample), can allow differentiation of patients (e.g., breast cancer patients) with a good prognosis from those patients with a bad prognosis.

In some embodiments, the assessment of a biological sample according to the methods disclosed herein can be used in combination with assessment of conventional clinical factors (e.g., tumor size, tumor grade, lymph node status, and family history) and/or analysis of the expression level of molecular markers, such as HER2 and estrogen and progesterone hormone receptors.

In some embodiments, the biomarkers of the disclosure are proteins whose overexpression is indicative of cancer prognosis, including those biomarkers involved in cell cycle regulation, DNA replication, transcription, signal transduction, cell proliferation, invasion, or metastasis. In some embodiments, the detection of overexpression of the biomarker proteins of the disclosure permits the evaluation of cancer prognosis and/or facilitates the separation of breast cancer patients based on cancer subtype, and/or into good and bad prognosis risk groups for the purposes of, for example, treatment selection.

Biomarker expression can be assessed at the protein or nucleic acid level. In some embodiments, immunohistochemistry techniques are provided that utilize antibodies to detect the expression of biomarker proteins in biological samples. In some embodiments, at least one antibody directed to a specific biomarker of interest may be used. In other embodiments, the expression of a biomarker can also be detected by nucleic acid-based techniques, including, for example, hybridization and RT-PCR.

In some embodiments, a patient in need of treatment can be identified based on the presence or absence of certain genetic markers. For example, in some embodiments, a patient can be identified based on the sequence of gene, or a mutation thereof.

In some embodiments, a patient in need of treatment can be identified based on the patient's haplotype. The term haplotype refers to a single-stranded segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific biomarkers, e.g., genetic markers, polymorphisms, and/or haplotypes can be accomplished by methods known in the art for detecting sequences at polymorphic sites. For example, standard techniques for genotyping for the presence of SNPs and/or microsatellite markers can be used, such as fluorescence-based techniques (Chen, X. et al., Genome Res. 9 (5): 492-98 (1999)), utilizing PCR, LCR, Nested PCR and other techniques for nucleic acid amplification, the disclosure of which is incorporated herein by reference in its entirety.

In some embodiments, specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave). Some of the available array platforms, including Affymetrix SNP Array 6.0 and Illumina CNV370-Duo and IM BeadChips, include SNPs that tag certain CNVs. This allows detection of CNVs via surrogate SNPs included in these platforms. Thus, by use of these or other methods available to the person skilled in the art, one or more alleles at polymorphic markers, including microsatellites, SNPs or other types of polymorphic markers, can be identified.

In some embodiments, biomarkers may be detected by sequencing technologies. Obtaining sequence information about an individual identifies particular nucleotides in the context of a sequence. For SNPs, sequence information about a single unique sequence site is sufficient to identify alleles at that particular SNP. For markers comprising more than one nucleotide, sequence information about the nucleotides of the individual that contain the polymorphic site identifies the alleles of the individual for the particular site. The sequence information can be obtained from a sample from the individual. In certain embodiments, the sample is a nucleic acid sample. In certain other embodiments, the sample is a protein sample.

Various methods for obtaining nucleic acid sequence are known to the skilled person, and all such methods are useful for practicing the disclosure. Sanger sequencing is a well-known method for generating nucleic acid sequence information. Recent methods for obtaining large amounts of sequence data have been developed, and such methods are also contemplated to be useful for obtaining sequence information. These include pyrosequencing technology (Ronaghi, M. et al. Anal Biochem 267:65-71 (1999); Ronaghi, et al., Biotechniques 25:876-878 (1998)), e.g. 454 pyrosequencing (Nyren, P., et al. Anal Biochem 208:171-175 (1993)), Illumina/Solexa sequencing technology (http://www.illumina.com; see also Strausberg, R L, et al Drug Disc Today 13:569-577 (2008)), and Supported Oligonucleotide Ligation and Detection Platform (SOLID) technology (Applied Biosystems, http://www.appliedbiosystems.com); Strausberg, R L, et al Drug Disc Today 13:569-577 (2008), the disclosures of which are incorporated herein by reference in their entireties.

In some embodiments, a patient in need of treatment can be identified based on the presence of one or more biomarkers, e.g., a genetic marker or haplotype, that puts them at risk for breast cancer, i.e., where at least one allele of at least one marker or haplotype is more frequently present in an individual at risk for the disease or trait (affected), or diagnosed with the disease or trait, compared to the frequency of its presence in a comparison group (control), such that the presence of the marker or haplotype is indicative of susceptibility to the disease or trait (e.g., breast cancer). The control group may in one embodiment be a population sample, i.e. a random sample from the general population. In another embodiment, the control group is represented by a group of individuals who are disease-free, i.e. individuals who have not been diagnosed with breast cancer. Such disease-free control may in one embodiment be characterized by the absence of one or more specific disease-associated symptoms.

In another embodiment, the disease-free control group is characterized by the absence of one or more disease-specific risk factors. Such risk factors are in one embodiment at least one environmental risk factor. Representative environmental factors are natural products, minerals or other chemicals which are known to affect, or contemplated to affect, the risk of developing the specific disease or trait. Other environmental risk factors are risk factors related to lifestyle, including but not limited to food and drink habits, geographical location of main habitat, and occupational risk factors. In another embodiment, the risk factors are at least one genetic risk factor.

Accordingly, an individual who is at an increased susceptibility (i.e., increased risk) for a disease, is an individual in whom at least one specific allele at one or more biomarkers, e.g., genetic markers or haplotype conferring increased susceptibility (increased risk) for the disease is identified (i.e., at-risk marker alleles or haplotypes). The at-risk marker or haplotype is one that confers an increased risk (increased susceptibility) of the disease. For example, significance associated with a marker or haplotype can be measured by a relative risk (RR). Alternatively, significance associated with a marker or haplotype can be measured using an odds ratio (OR).

In some embodiments, significance can be measured by a percentage. In another embodiment, a significant increased risk is measured as a risk (relative risk and/or odds ratio) of at least 1.2, including but not limited to: at least 1.2, at least 1.3, at least 1.4, at least 1.5, at least 1.6, at least 1.7, 1.8, at least 1.9, at least 2.0, at least 2.5, at least 3.0, at least 4.0, and at least 5.0. In a particular embodiment, a risk (relative risk and/or odds ratio) of at least 1.2 is significant. In another particular embodiment, a risk of at least 1.3 is significant. In yet another embodiment, a risk of at least 1.4 is significant. In a further embodiment, a relative risk of at least 1.5 is significant. In another further embodiment, a significant increase in risk is at least 1.7 is significant. However, other cutoffs are also contemplated, e.g., at least 1.15, 1.25, 1.35, and so on, and such cutoffs are also within scope of the present disclosure. In other embodiments, a significant increase in risk is at least about 20%, including but not limited to about 25%, 30%, 35%, 40%, 45%, 50%, 55%, 60%, 65%, 70%, 75%, 80%, 85%, 90%, 95%, 100%, 150%, 200%, 300%, and 500%. In one particular embodiment, a significant increase in risk is at least 20%. In other embodiments, a significant increase in risk is at least 30%, at least 40%, at least 50%, at least 60%, at least 70%, at least 80%, at least 90% and at least 100%. Other cutoffs or ranges as deemed suitable by the person skilled in the art to characterize the disclosure are however also contemplated, and those are also within scope of the present disclosure. In certain embodiments, a significant increase in risk is characterized by a p-value, such as a p-value of less than 0.05, less than 0.01, less than 0.001, less than 0.0001, less than 0.00001, less than 0.000001, less than 0.0000001, less than 0.00000001, or less than 0.000000001.

An exemplary method for assessing SNPs, genetic variants, identifying patients susceptible to breast cancer, calculating risk, and the like, is described in U.S. Pat. No. 8,951,735, the disclosure of which is incorporated herein by reference in its entirety.

In some embodiments, a patient in need of treatment can be identified based on the presence of one or more biomarkers, wherein the biomarker is one or more polymorphic variants associated with breast cancer.

An exemplary description of polymorphic variants associated with breast cancer risk is provided in U.S. Pat. No. 7,510,835, the disclosure of which is incorporated herein by reference in its entirety.

Examples of cell cycle, proliferation, and cancer biomarkers, along with methods of detecting the same, in providing a prognosis for breast cancer are provided in EP1800130B1, the disclosure of which is incorporated herein by reference in its entirety.

### Methods for Detecting Cancer Biomarker Levels a Biological Sample

A patient and/or a biological sample isolated therefrom can be assessed and/or evaluated by detecting a level of a biomarker (e.g., whether a breast cancer tumor comprises HRD; and whether the tumor is ER+; HER2−; and/or PR+), in a biological sample, such as a biopsy, obtained from the patient (e.g., a patient having cancer that is in need of treatment).

In some embodiments, the biological sample can be any type of biological sample described herein, including, without limitation, cells, tissue (e.g., a tissue sample obtained by biopsy), blood, serum, plasma, urine, sputum, cerebrospinal fluid, lymph tissue or fluid, or pancreatic fluid. For example, in some embodiments, the biological sample can be fresh frozen or formalin-fixed paraffin embedded (FFPE) tissue obtained from the subject, such as a tumor sample (e.g., a biopsy) from the tissue of interest (e.g., prostate, ovarian, lung, lymph nodes, thymus, spleen, bone marrow, breast, colorectal, pancreatic, cervical, bladder, gastrointestinal, head, or neck tissue).

Methods of detecting biomarker levels in a biological sample are discussed in detail below.

### RNA Extraction and Measurement of Biomarker Levels

In some embodiments, a biological sample, e.g., cell samples or tissue samples, may be snap frozen in liquid nitrogen until processing. RNA may be extracted using, e.g., Trizol Reagent from Invitrogen following manufacturer's instructions, and detected directly or converted to cDNA for detection. RNA may be amplified using, e.g., MessageAmp kit from Ambion following manufacturer's instructions. Amplified RNA may be quantified using, e.g., HG-U133A or HG-U133_Plus2 GeneChip from Affymetrix Inc. or a compatible apparatus, e.g., the GCS3000Dx GeneChip® System from Affymetrix Inc., according to the manufacturer's instructions. The resulting biomarker level measurements may be further analyzed as described herein. In some embodiments, analysis of biomarker levels can be implemented using, e.g., R software available from R-Project (http://www.r-project.org) and supplemented with packages available from Bioconductor (http://www.bioconductor.org).

In some embodiments, the level of one or more of the biomarkers described herein, may be measured in a biological sample (e.g., a tumor sample) obtained from the patient (e.g., a patient having any of the cancer types described herein, such as a patient having breast cancer) using, e.g., polymerase chain reaction (PCR), reverse transcriptase PCR (RT-PCR), quantitative real-time PCR (qRT-PCR), an array (e.g., a microarray), a genechip, pyrosequencing, nanopore sequencing, sequencing by synthesis, sequencing by expansion, single molecule real time technology, sequencing by ligation, microfluidics, infrared fluorescence, next generation sequencing (NGS) (e.g., RNA-Seq techniques), Northern blots, Western blots, Southern blots, NanoString nCounter technologies (e.g., those described in U.S. Patent Application Nos. US 2011/0201515, US 2011/0229888, and US 2013/0017971, each of which is incorporated by reference in its entirety), proteomic techniques (e.g., mass spectrometry or protein arrays), and combinations thereof.

### Assessing Biological Samples and Patient Screening

The present disclosure provides methods that comprise assessing a patient, assessing a biological sample, evaluating a biological sample, determining the status of biological sample (e.g., whether a breast cancer tumor comprises HRD; and whether the tumor is ER+; HER2−; and/or PR+), and/or diagnosing a patient based on the information obtained from a biological sample.

Diagnosis of a patient and/or a biological sample derived therefrom, may be performed by any method or technique known in the art. For example, in addition to standard molecular biology techniques described herein, techniques such as x-ray and MRI may be employed. In some embodiments, the results of any analysis described herein may be further confirmed by a physician.

In another aspect of the present disclosure, methods are provided to identify cancers or tumors that are susceptible to the inhibitory effects, or are responsive, to the compounds of the present disclosure.

In various embodiments, the present disclosure provides a method for preventing and treating a breast cancer in a human subject, wherein the breast cancer tumor comprises HRD, and the breast cancer tumor is ER positive, the method comprising administering a therapeutically effective amount of a PARG modulator of the present disclosure represented in Formula (I), or a pharmaceutically acceptable salt thereof. In related embodiments, the breast cancer tumor comprises HRD, is ER positive and optionally HER2−, and optionally PR positive.

In some embodiments, methods are provided to treat a cancer or tumor in a mammal by (i) obtaining a biological sample from the tumor; (ii) determining whether the cancer or tumor comprises cells that HRD and are ER+; and (iii) treating the cancer or tumor with one or more of the compounds described herein.

In some embodiments, methods are provided to treat a HRD and an ER+ cancer or tumor in a mammal by treating the cancer or tumor with one or more of the compounds described herein.

In one embodiment, the biological sample can be a biopsy. In other embodiments, the biological sample can be fluid, cells and/or aspirates obtained from the tumor or cancer.

In some embodiments, the biopsy can be a tumor biopsy, a liquid biopsy (e.g., obtained from peripheral blood).

The biological sample can be obtained according to any technique known to one skilled in the art. In one embodiment, a biopsy can be conducted to obtain the biological sample. A biopsy is a procedure performed to remove tissue or cells from the body for examination. Some biopsies can be performed in a physician's office, while others need to be done in a hospital setting. In addition, some biopsies require use of an anesthetic to numb the area, while others do not require any sedation.

In certain embodiments, an endoscopic biopsy can be performed. This type of biopsy is performed through a fiberoptic endoscope (a long, thin tube that has a close-focusing telescope on the end for viewing) through a natural body orifice (i.e., rectum) or a small incision (i.e., arthroscopy). The endoscope is used to view the organ in question for abnormal or suspicious areas, in order to obtain a small amount of tissue for study. Endoscopic procedures are named for the organ or body area to be visualized and/or treated. The physician can insert the endoscope into the gastrointestinal tract (alimentary tract endoscopy), bladder (cystoscopy), abdominal cavity (laparoscopy), joint cavity (arthroscopy), mid-portion of the chest (mediastinoscopy), or trachea and bronchial system (laryngoscopy and bronchoscopy).

In another embodiment, a bone marrow biopsy can be performed. This type of biopsy can be performed either from the sternum (breastbone) or the iliac crest hipbone (the bone area on either side of the pelvis on the lower back area). The skin is cleansed and a local anesthetic is given to numb the area. A long, rigid needle is inserted into the marrow, and cells are aspirated for study; this step is occasionally uncomfortable. A core biopsy (removing a small bone ‘chip’ from the marrow) may follow the aspiration.

In a further embodiment, an excisional or incisional biopsy can be performed on the mammal. This type of biopsy is often used when a wider or deeper portion of the skin is needed. Using a scalpel (surgical knife), a full thickness of skin is removed for further examination, and the wound is sutured (sewed shut with surgical thread). When the entire tumor is removed, it is referred to as an excisional biopsy technique. If only a portion of the tumor is removed, it is referred to as an incisional biopsy technique. Excisional biopsy is often the method usually preferred, for example, when melanoma (a type of skin cancer) is suspected.

In still further embodiments, a fine needle aspiration (FNA) biopsy can be used. This type of biopsy involves using a thin needle to remove very small pieces from a tumor. Local anesthetic is sometimes used to numb the area, but the test rarely causes much discomfort and leaves no scar. FNA is not, for example, used for diagnosis of a suspicious mole, but may be used, for example, to biopsy large lymph nodes near a melanoma to see if the melanoma has metastasized (spread). A computed tomography scan (CT or CAT scan) can be used to guide a needle into a tumor in an internal organ such as the lung or liver.

In other embodiments, punch shave and/or skin biopsies can be conducted. Punch biopsies involve taking a deeper sample of skin with a biopsy instrument that removes a short cylinder, or “apple core,” of tissue. After a local anesthetic is administered, the instrument is rotated on the surface of the skin until it cuts through all the layers, including the dermis, epidermis, and the most superficial parts of the subcutis (fat). A shave biopsy involves removing the top layers of skin by shaving it off. Shave biopsies are also performed with a local anesthetic. Skin biopsies involve removing a sample of skin for examination under the microscope to determine if, for example, melanoma is present. The biopsy is performed under local anesthesia.

In some embodiments, the biological sample can be circulating tumor DNA (ctDNA).

Exemplary methods regarding circulating tumor DNA are provided in: U.S. Patent Application Nos: US20180024141A1; US20180196049A1; US20190107542A1; US20200191793 A1; and U.S. Pat. No. 8,329,422; the disclosures of which are incorporated herein by reference in their entireties.

In some embodiments, a biological sample can be assessed and/or evaluated using circulating tumor cell (CTC) analysis.

Exemplary methods of CTC analysis are provided in: P. Bossolasco, C. Ricci, G. Farina et al., “Detection of micrometastatic cells in breast cancer by RT-PCR for the mammaglobin gene,” Cancer Detection and Prevention, vol. 26, no. 1, pp. 60-63, 2002; V. V. Iakovlev, R. S. Goswami, J. Vecchiarelli, N. C. R. Arneson, and S. J. Done, “Quantitative detection of circulating epithelial cells by Q-RT-PCR,” Breast Cancer Research and Treatment, vol. 107, no. 1, pp. 145-154, 2008; N. Xenidis, M. Perraki, M. Kafousi et al., “Predictive and prognostic value of peripheral blood cytokeratin-19 mRNA-positive cells detected by real-time polymerase chain reaction in node-negative breast cancer patients,” Journal of Clinical Oncology, vol. 24, no. 23, pp. 3756-3762, 2006; N. Cabioglu, A. Igci, E. O. Yildirim et al., “An ultrasensitive tumor enriched flow-cytometric assay for detection of isolated tumor cells in bone marrow of patients with breast cancer,” American Journal of Surgery, vol. 184, no. 5, pp. 414-417, 2002; I. Cruz, J. Ciudad, J. J. Cruz et al., “Evaluation of multiparameter flow cytometry for the detection of breast cancer tumor cells in blood samples,” American Journal of Clinical Pathology, vol. 123, no. 1, pp. 66-74, 2005; S. J. Simpson, M. Vachula, M. J. Kennedy et al., “Detection of tumor cells in the bone marrow, peripheral blood, and apheresis products of breast cancer patients using flow cytometry,” Experimental Hematology, vol. 23, no. 10, pp. 1062-1068, 1995; S. Braun, F. D. Vogl, B. Naume et al., “A pooled analysis of bone marrow micrometastasis in breast cancer,” The New England Journal of Medicine, vol. 353, no. 8, pp. 793-802, 2005; K. Pachmann, O. Camara, A. Kavallaris et al., “Monitoring the response of circulating epithelial tumor cells to adjuvant chemotherapy in breast cancer allows detection of patients at risk of early relapse,” Journal of Clinical Oncology, vol. 26, no. 8, pp. 1208-1215, 2008; A. E. Ring, L. Zabaglo, M. G. Ormerod, I. E. Smith, and M. Dowsett, “Detection of circulating epithelial cells in the blood of patients with breast cancer: comparison of three techniques,” British Journal of Cancer, vol. 92, no. 5, pp. 906-912, 2005; L. Zabaglo, M. G. Ormerod, M. Parton, A. Ring, I. E. Smith, and M. Dowsett, “Cell filtration-laser scanning cytometry for the characterisation of circulating breast cancer cells,” Cytometry A, vol. 55, no. 2, pp. 102-108, 2003; M. Cristofanilli, G. T. Budd, M. J. Ellis et al., “Circulating tumor cells, disease progression, and survival in metastatic breast cancer,” The The New England Journal of Medicine, vol. 351, no. 8, pp. 781-791, 2004; S. Riethdorf, H. Fritsche, V. Muller et al., “Detection of circulating tumor cells in peripheral blood of patients with metastatic breast cancer: a validation study of the cell search system,” Clinical Cancer Research, vol. 13, no. 3, pp. 920-928, 2007; S. Nagrath, L. V. Sequist, S. Maheswaran et al., “Isolation of rare circulating tumour cells in cancer patients by microchip technology,” Nature, vol. 450, no. 7173, pp. 1235-1239, 2007; A. G. J. Tibbe, M. C. Miller, and L. W. Terstappen, “Statistical considerations for enumeration of circulating tumor cells,” Cytometry A, vol. 71, no. 3, pp. 154-162, 2007; the disclosures of which are incorporated herein by reference in their entireties.

The biological sample can be assessed, evaluated, using any method described herein or known in the art. Further, determining the status of biological sample (e.g., whether a breast cancer tumor comprises HRD; and whether the tumor is ER+; HER2−; PR+, and/or diagnosing a patient based on the information obtained from a biological sample, can likewise be performed using any method described herein and/or known in the art.

In some embodiments, the present disclosure comprises a method of treating breast cancer in a patient in need thereof, the method comprising: (a) selecting a patient having a breast cancer tumor wherein the tumor comprises HRD, wherein said tumor is an estrogen receptor (ER) positive (ER+) tumor; and (b) administering to the patient in need thereof, a therapeutically effective amount of a Poly(ADP-ribose) glycohydrolase (PARG) modulator; and further comprising assessing the biological sample, or evaluating the information about the biological sample, to determine whether the tumor: (a) comprises HRD; (b) is a ER+ tumor; and (c) is optionally a HER2-tumor; and (d) is optionally a PR+ tumor.

In some embodiments, assessing the biological sample, and/or determining the status and/or quality of the breast cancer tumor, can be performed using any method known to those in the art, or described herein. For example, in some embodiments, the biological sample can be assessed using Next-generation sequencing (NGS), single-cell analysis of circulating tumor cells (CTC), CTC genome sequencing, ctDNA analysis, and other methods known by those having skill in the art, and/or described herein.

In some embodiments, CTC analysis can be performed using PCR, flow cytometry, image-based immunologic approaches, immunomagnetic techniques, and/or microchip technology.

In some embodiments, CTC analysis can be performed as described herein.

In some embodiments, the present disclosure comprises a method of treating breast cancer in a patient in need thereof, the method comprising: (a) selecting a patient having a breast cancer tumor wherein the tumor comprises HRD, and is an estrogen receptor (ER) positive (ER+) tumor; and (b) administering to the patient in need thereof, a therapeutically effective amount of a Poly(ADP-ribose) glycohydrolase (PARG) modulator; and further comprising: obtaining multiple biological samples, or obtaining information about multiple biological samples, of the breast cancer tumor; wherein the multiple biological samples are taken at separate points of time; and assessing the multiple biological samples, or evaluating the information about multiple biological samples to determine whether the breast cancer tumor: (a) comprises HRD; (b) is an ER+ tumor; and (c) is optionally a HER2−; and (d) is optionally a PR+ tumor.

In some embodiments, the present disclosure comprises a method of treating breast cancer in a patient in need thereof, the method comprising: (a) selecting a patient having a breast cancer tumor wherein in the tumor comprises HRD, is an estrogen receptor (ER) positive (ER+) tumor; and (b) administering to the patient in need thereof, a therapeutically effective amount of a Poly(ADP-ribose) glycohydrolase (PARG) modulator; and further comprising: obtaining multiple biological samples, or obtaining information about multiple biological samples, of the breast cancer tumor; wherein the multiple biological samples are taken at separate points of time and/or from spacially discrete regions of the breast cancer tumor; and assessing the multiple biological samples, or evaluating the information about multiple biological samples to determine whether the breast cancer tumor: (a) comprises HRD; (b) is an ER+ tumor; and (c) is optionally a HER2−; and (d) is optionally a PR+ tumor.

In some embodiments, the present disclosure comprises a method of treating breast cancer in a patient in need thereof, the method comprising: (a) selecting a patient having a breast cancer tumor wherein the tumor comprises HRD, and is an estrogen receptor (ER) positive (ER+) tumor; and (b) administering to the patient in need thereof, a therapeutically effective amount of a Poly(ADP-ribose) glycohydrolase (PARG) modulator; and further comprising assessing the biological sample, or evaluating the information about the biological sample, to determine whether the breast cancer tumor: (a) comprises HRD; (b) is a ER+ tumor; and (c) is optionally a HER2-tumor; and (d) is optionally a PR+ tumor.

In some embodiments, the present disclosure comprises a method of treating breast cancer in a patient in need thereof, the method comprising: (a) obtaining a biological sample from the patient; (b) detecting a level of one or more biomarkers; and (c) comparing the level of the one or more biomarkers in the patient relative to a control.

### Determining HRD, ER, HER2, and PR Tumor Status

Determining the status of a tumor, e.g., whether said tumor comprises HRD and/or whether said tumor is ER+; PR+; and/or HER2−, can be performed using any technique known to those in the art.

### ER+ Tumor Status

In some embodiments, a tumor may be ER+, i.e., in some embodiments, a breast cancer may have estrogen receptors.

In some embodiments, estrogen receptor positive (ER+) tumor status is determined when the tumor cells score positive, i.e., using conventional histopathology methods, for the estrogen receptor (ER). In some embodiments, a tumor is ER+ if at least 1% of the tumor cells tested (e.g., by immunohistochemistry) score ER positive. In some embodiments, a tumor is ER+ if at least 10% of the tumor cells tested (e.g., by immunohistochemistry) score ER positive.

In some embodiments, determination of ER status is as follows: tumor biopsies will be processed into FFPE blocks prior to immunohistochemistry (IHC) and slides will be stained with antibodies against the estrogen receptor (ER). Antibody-specific staining for ER is scored manually by a board-certified pathologist and/or at a contracted external good clinical practice-accredited laboratory. Next, ER status can be evaluated by percentage of nuclear staining and considered positive when there was ≥1% nuclear staining. The percentage of tumor cells can also be assessed with an intensity of negative, weak, moderate, and strong staining (0, negative; 1, weak; 2, moderate; 3, strong). Results for ER will be recorded as an H score [sum of (1×percentage weak) [symbol] (2×percentage moderate) [symbol] (3×percentage strong)] to a maximum of 300.

In some embodiments a ER+ tumor sample is, e.g., a biopsy that is evaluated using any of the methods described herein (e.g., IHC, PCR, etc.), wherein said biopsy has cancer cells therein that are positive for estrogen receptors.

In some embodiments, a biological sample with 1% to 100% of tumor nuclei positive for ER are interpreted as positive. In some embodiments, if 1% to 10% of tumor cell nuclei are immunoreactive, the sample should be reported as ER Low Positive. In some embodiments, a biological sample may be considered negative for ER if <1% or 0% of tumor cell nuclei are immunoreactive. In some embodiments, a biological sample may be deemed uninterpretable for ER if the sample is inadequate (e.g., insufficient cancer or severe artifacts present, as determined at the discretion of the pathologist).

### PR+ Tumor Status

In some embodiments, a tumor may be PR+, i.e., in some embodiments, a breast cancer may have progesterone receptors.

In some embodiments, determination of PR status is as follows: tumor biopsies will be processed into FFPE blocks prior to immunohistochemistry (IHC) and slides will be stained with antibodies against the progesterone receptor (PR). Antibody-specific staining for PR is scored manually by a board-certified pathologist and/or at a contracted external good clinical practice-accredited laboratory. PR status can be evaluated by percentage of nuclear staining and considered positive when there is ≥1% nuclear staining. The percentage of tumor cells can also be assessed with an intensity of negative, weak, moderate, and strong staining (0, negative; 1, weak; 2, moderate; 3, strong). Results for PR may be recorded as an H score [sum of (1×percentage weak) [symbol] (2×percentage moderate) [symbol] (3×percentage strong)] to a maximum of 300.

In some embodiments a PR+ tumor sample is, e.g., a biopsy that is evaluated using any of the methods described herein (e.g., IHC, PCR, etc.), wherein said biopsy has cancer cells therein that are positive for progesterone receptors.

In some embodiments, a biological sample with 1% to 100% of tumor nuclei positive for PR are interpreted as positive. In some embodiments, a biological sample with at least 10% of tumor nuclei positive for PR are interpreted as positive. In some embodiments, a biological sample may be considered negative for PR if <1% or 0% of tumor cell nuclei are immunoreactive. In some embodiments, a biological sample may be deemed uninterpretable for PR if the sample is inadequate (e.g., insufficient cancer or severe artifacts present, as determined at the discretion of the pathologist).

### HER2− Tumor Status

In some embodiments, a tumor designated HER2 negative (HER2−) is a tumor in which an immunoassay such as immunohistochemistry (IHC) test shows no staining or membrane staining in <30% of tumor cells. In some embodiments, a commercial assay can be used to determine HER2 tumor status, e.g., HERCEPTEST®, wherein a score of 0 or 1+ is considered HER2 negative, a score of 2+ is considered equivocal—requiring further testing by fluorescence in-situ hybridization (FISH) for definitive characterization, and a score of 3+ is considered HER2 positive. Therefore a patient with a biopsy scoring 0 or 1+ by HERCEPTEST, or 2+ by HERCEPTEST and negative by FISH is considered HER2 negative, while a patient scoring 3+ by HERCEPTEST or 2+ by HERCEPTEST and FISH positive is deemed HER2 positive.

### Immunohistochemistry

In some embodiments, immunohistochemistry can be used to determine the status of a biological sample (e.g., a tumor biopsy). Immunohistochemistry is a procedure for determining the presence or distribution of an antigen in a sample by detecting the interaction of the antigen with a specific binding agent, such as an antibody. A sample is contacted with an antibody detected by means of a detectable label conjugated with the antibody (direct detection) or by means of a detectable label conjugated with a secondary antibody, which specifically binds to the primary antibody (e.g. indirect detection).

### In Situ Hybridization (ISH)

In situ hybridization is a procedure to determine the presence or distribution of a nucleic acid in a sample using hybridization of a labeled nucleic acid probe to locate a specific DNA or RNA sequence in a portion or section of tissue (in situ) or, if the tissue is small enough (for example, vegetable seeds, Drosophila embryos), throughout the tissue (ISH full preparation). DNA ISH can be used to determine the structure of chromosomes, such as for use in medical diagnostics to assess chromosomal integrity and/or determine the number of copies of the gene in a sample. RNA ISH measures and locates mRNAs and other transcripts within histological sections or complete preparations.

For ISH, sample cells and tissues are usually treated to fix the target nucleic acids in place and to increase the access of the probe to the target molecule. The detectably labeled probe hybridizes with the target sequence at elevated temperature, and then the excess probe is removed by washing. The solution parameters, such as temperature, salt concentration and/or detergent, can be manipulated to eliminate any non-identical interaction (for example, so that only the exact sequence matches remain attached). Then, the labeled probe is located and potentially quantified in the tissue using either autoradiography, fluorescence microscopy or immunohistochemistry, respectively. ISH can also use two or more probes, which are typically marked differently to simultaneously detect two or more nucleic acids.

ISH methods are provided in Sambrook et al. (in Molecular Cloning: A Laboratory Manual, CSHL, New York, 1989) and Ausubel et al. (in Current Protocols in Molecular Biology, Greene Publ. Assoc, and Wiley-Intersciences, 1992), the disclosure of which is incorporated herein by reference in its entirety.

### Administration

In some embodiments, a therapeutically effective amount of one or more of the compounds of the present disclosure can be administered to a subject in need thereof.

In some embodiments, a therapeutically effective amount is an amount sufficient to prevent or delay recurrence of a disease, (e.g., cancer). In some embodiments, the therapeutically effective amount can be administered in one or more administrations. The therapeutically effective amount of the drug or combination may result in one or more of the following: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

The present disclosure contemplates any route of administration. For example, in some embodiments, the route of administration can refer to oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration can be by any route, including parenteral and transmucosal (e.g., buccal, sublingual, palatal, gingival, nasal, vaginal, rectal, or transdermal).

### Non-Limiting Exemplary Embodiments

In further embodiments 1 to 45 below, the present disclosure includes:

1. In embodiment 1, provided is a method of treating breast cancer in a patient in need thereof, the method comprising:
(a) selecting a patient having a breast cancer tumor wherein the tumor comprises homologous recombination deficiency (HRD) and is estrogen receptor (ER) positive (ER+); and
(b) administering to the patient in need thereof, a therapeutically effective amount of a Poly(ADP-ribose) glycohydrolase (PARG) modulator.

2. In embodiment 2, the method of embodiment 1, wherein the breast cancer tumor is a human epidermal growth factor receptor-2 (HER2) negative (HER2−) tumor.

3. In embodiment 3, the method of embodiment 1 or 2, wherein the breast cancer tumor is a progesterone receptor (PR) positive (PR+) tumor.

4. In embodiment 4, the method of any one of embodiments 1 to 3, wherein the PARG modulator is a small molecule PARG inhibitor.

In embodiment 5, the method of any one of embodiments 1 to 4, wherein the PARG modulator is a compound of Formula (I):

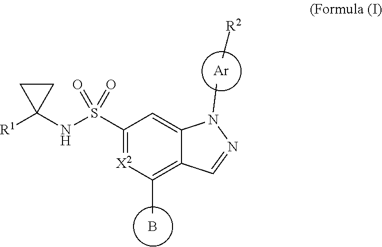

or a pharmaceutically acceptable salt thereof, wherein
R¹ is selected from the group consisting of cyano, C₁₋₂ alkyl, and C₁₋₂ haloalkyl;
Ar is a 5-membered heteroaryl;
X² is CH or CF;
R² is selected from the group consisting of C₁₋₃ alkyl, C₁₋₃ haloalkyl, hydroxyC₁₋₃alkyl, and cyano;
ring B is 5- or 6-membered heterocycloalkyl substituted with R^{a}, R^{b}, and R^{c};
R^{a} is hydrogen, C₁₋₄ alkyl, C₁₋₄ haloalkyl, halo, hydroxy, —C(O)R^{d} (where R^{d} is hydrogen, C₁₋₆ alkyl, or C₁₋₆ haloalkyl); and
R^{b} and R^{c} are independently selected from hydrogen, C₁₋₆ alkyl, hydroxy, C₁₋₆ alkoxy, halo, C₁₋₆ haloalkyl, and C₁₋₆ haloalkoxy.

6. In embodiment 6, the compound of embodiment 5, or a pharmaceutically acceptable salt thereof wherein X² is CH.

7. In embodiment 7, the compound of embodiment 5 or 6, or a pharmaceutically acceptable salt thereof wherein R¹ is cyano.

8. In embodiment 8, the compound of embodiment 5 or 6, or a pharmaceutically acceptable salt thereof wherein R¹ is methyl or ethyl.

9. In embodiment 9, the compound of embodiment 5 or 6, or a pharmaceutically acceptable salt thereof wherein R¹ is methyl.

10. In embodiment 10, the compound of any one of embodiments 5 to 9, or a pharmaceutically acceptable salt thereof, wherein Ar is 1,2,4-thiadiazolyl, or 1,3,4-thiadiazolyl.

11. In embodiment 11, the compound of any one of embodiments 5 to 10, or a pharmaceutically acceptable salt thereof, wherein Ar is 1,2,4-thiadiazolyl.

12. In embodiment 12, the compound of any one of embodiments 5 to 10, or a pharmaceutically acceptable salt thereof, wherein Ar is 1,3,4-thiadiazolyl.

13. In embodiment 13, the compound of any one of embodiments 5 to 12, or a pharmaceutically acceptable salt thereof, wherein R² is attached to the carbon atom of Ar that is meta to the atom of Ar that is attached to the nitrogen atom of the remainder of the molecule.

14. In embodiment 14, the compound of any one of embodiments 5 to 13, or a pharmaceutically acceptable salt thereof, wherein R² is methyl, ethyl, difluoromethyl, trifluoromethyl, cyano.

15. In embodiment 15, the compound of any one of embodiments 5 to 14, or a pharmaceutically acceptable salt thereof, wherein R² is difluoromethyl.

16. In embodiment 16, the compound of any one of embodiments 5 to 15, or a pharmaceutically acceptable salt thereof, wherein ring B is morpholinyl, 1,1-dioxothiomorpholinyl, pyrrolidinyl, piperidinyl, 6-oxo-1,6-dihydropyridinyl, or piperazinyl.

17. In embodiment 17, the compound of any one of embodiments 5 to 16, or a pharmaceutically acceptable salt thereof, wherein ring B is piperazinyl.

18. In embodiment 18, the compound of any one of embodiments 5 to 17, or a pharmaceutically acceptable salt thereof, wherein R^{a} is hydrogen, C₁₋₄ alkyl, C₁₋₄ haloalkyl, halo, hydroxy, —C(O)R^{d} (where R^{d} is hydrogen, C₁₋₆ alkyl, or C₁₋₆ haloalkyl); and R^{b} and R^{c} are independently selected from hydrogen, C₁₋₆ alkyl, hydroxy, C₁₋₆ alkoxy, halo, C₁₋₆ haloalkyl, and C₁₋₆ haloalkoxy.

19. In embodiment 19, the compound of any one of embodiments 5 to 18, or a pharmaceutically acceptable salt thereof, wherein R^{a} is hydrogen, C₁₋₄ alkyl, C₁₋₄ haloalkyl, —C(O)R^{d} (where R^{d} is C₁₋₆ alkyl, or C₁₋₆ haloalkyl); and R^{b} and R^{c} are independently selected from hydrogen, C₁₋₆ alkyl, and C₁₋₆ haloalkoxy.

20. In embodiment 20, the compound of any one of embodiments 5 to 19, or a pharmaceutically acceptable salt thereof, wherein R^{a} is —C(O)R^{d} where R^{d} is C₁₋₆ alkyl; and R^{b} and R^{c} are independently selected from hydrogen, C₁₋₆ alkyl, and C₁₋₆ haloalkoxy.

21. In embodiment 21, the compound of any one of embodiments 5 to 19, or a pharmaceutically acceptable salt thereof, wherein R^{a} is hydrogen C₁₋₄ alkyl, and C₁₋₄ haloalkyl; and R^{b} and R^{c} are independently selected from hydrogen, and C₁₋₆ alkyl.

22. In embodiment 22, the compound of any one of embodiments 5 to 21, or a pharmaceutically acceptable salt thereof, wherein the compound is selected from the group consisting of:

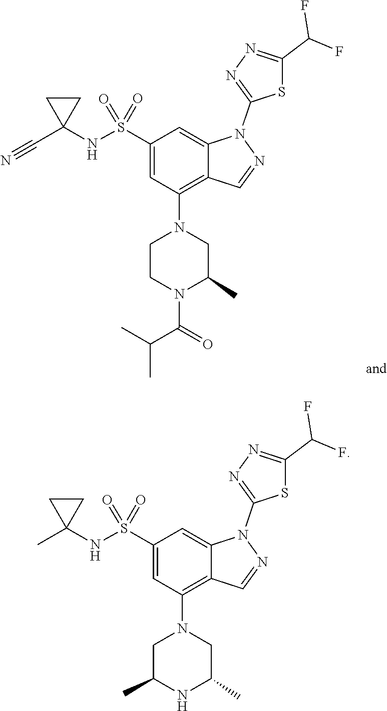 [SMILES: CC(C)C(=O)N1CCN(c2cc(S(=O)(=O)NC3(C#N)CC3)cc3c2cnn3-c2nnc(C(F)F)s2)C[C@H]1C ; C[C@H]1CN(c2cc(S(=O)(=O)NC3(C)CC3)cc3c2cnn3-c2nnc(C(F)F)s2)C[C@H](C)N1]

23. In embodiment 23, the method of any one of embodiments 1 to 22, further comprising obtaining a biological sample from the breast cancer tumor, or obtaining information about the biological sample.

24. In embodiment 24, the method of embodiment 23, further comprising assessing the biological sample, or evaluating the information about the biological sample, to determine whether the breast cancer tumor comprises:
(a) homologous recombination deficiency (HRD); and
(b) is ER+.

25. In embodiment 25, the method of embodiment 24, further comprising assessing the biological sample, or evaluating the information about the biological sample, to determine whether the breast cancer tumor is HER2-tumor.

26. In embodiment 26, the method of embodiment 24 or 25, further comprising the biological sample, or evaluating the information about the biological sample, to determine whether the breast cancer tumor is PR+ tumor.

27. In embodiment 27, the method of any one of embodiments 23 to 26, further comprising obtaining multiple biological samples, or obtaining information about multiple biological samples, of the breast cancer tumor; wherein the multiple biological samples are taken at separate points of time; and assessing the multiple biological samples, or evaluating the information about multiple biological samples to determine whether the breast cancer tumor comprises:
(a) homologous recombination deficiency (HRD); and
(b) is ER+.

28. In embodiment 28, the method of embodiment 27, further comprising obtaining multiple biological samples, or obtaining information about multiple biological samples, of the breast cancer tumor; wherein the multiple biological samples are taken at separate points of time; and assessing the multiple biological samples, or evaluating the information about multiple biological samples to determine whether the breast cancer tumor is HER2-tumor.

29. In embodiment 29, the method of embodiment 27 or 28, further comprising obtaining multiple biological samples, or obtaining information about multiple biological samples, of the breast cancer tumor; wherein the multiple biological samples are taken at separate points of time; and assessing the multiple biological samples, or evaluating the information about multiple biological samples to determine whether the breast cancer tumor is PR+ tumor.

30. In embodiment 30, the method of any one of embodiments 23 to 29, wherein the biological sample is a biopsy

31. In embodiment 31, the method of embodiment 30, wherein the biopsy is a tumor biopsy, or a liquid biopsy.

32. In embodiment 32, the method of any one embodiments 23 to 31, wherein the biological sample is a tumor biopsy obtained by core needle biopsy.

33. In embodiment 33, the method of any one of embodiments 23 to 31, wherein the biological sample is a tumor biopsy prepared in the form of tissue sections.

34. In embodiment 34, the method of embodiment 33, wherein the tissue sections are fixed and paraffin-embedded.

35. In embodiment 35, the method of any one of embodiments 23 to 34, wherein the biological sample is frozen.

36. In embodiment 36, the method of any one of embodiments 23 to 35, wherein the biological sample is prepared in the form of isolated cells, lysed cells, homogenate, cell fraction, or a combination thereof.

37. In embodiment 37, the method of embodiment 36, wherein the biological sample is cultured cells.

38. In embodiment 38, the method of any one of embodiments 1 to 37, wherein the homologous recombination deficiency (HRD) is characterized by HRD score, HRDetect, single base substitution signature 3 (SBS3), or indel signature 6 (ID6).

39. In embodiment 39, the method of any one of embodiments 24 to 38, wherein assessing the biological sample or evaluating the information about the biological sample comprises identifying homologous recombination deficiency (HRD) in DNA extracted from a tumor sample.

40. In embodiment 40, the method of any one of embodiments 24 to 39, wherein assessing the biological sample or evaluating the information about the biological sample comprises identifying homologous recombination deficiency (HRD) in a circulating tumor DNA.

41. In embodiment 41, the method of any one of embodiments 23 to 40, wherein assessing the biological sample, or evaluating the information about the biological sample comprises determining whether the breast cancer tumor is ER+ tumor.

42. In embodiment 42, the method of any one of embodiments 23 to 41, wherein assessing the biological sample, or evaluating the information about the biological sample comprises determining whether the breast cancer tumor is HER2-tumor.

43. In embodiment 43, the method of any one of embodiments 23 to 41, wherein assessing the biological sample, or evaluating the information about the biological sample comprises determining whether the breast cancer tumor is PR+ tumor.

44. In embodiment 44, the method of any one of embodiments 1 to 43, wherein the PARG modulator is administered to the patient as a monotherapy.

45. In embodiment 45, the method of any one embodiments 1 to 44, further comprising administering at least one additional therapeutic agent.

### Additional Non-Limiting Exemplary Embodiments

In further embodiments 1 to 51 below, the present disclosure includes:

1. In embodiment 1, provided is a method of treating breast cancer in a patient in need thereof, the method comprising:
(a) selecting a patient having a breast cancer tumor wherein the tumor comprises homologous recombination deficiency (HRD) and is estrogen receptor (ER) positive (ER+); and
(b) administering to the patient in need thereof, a therapeutically effective amount of a Poly(ADP-ribose) glycohydrolase (PARG) modulator.

1A. In embodiment 1A, provided is a method of treating breast cancer in a patient in need thereof, the method comprising administering a therapeutically effective amount of a a Poly(ADP-ribose) glycohydrolase (PARG) modulator to the patient in need thereof, wherein the patient has a breast cancer tumor and the tumor comprises homologous recombination deficiency (HRD) and is estrogen receptor (ER) positive (ER+).

2. In embodiment 2, the method of embodiment 1 or 1A, wherein the breast cancer tumor is a human epidermal growth factor receptor-2 (HER2) negative (HER2−) tumor.

3. In embodiment 3, the method of embodiments 1, 1A, or 2, wherein the breast cancer tumor is a progesterone receptor (PR) positive (PR+) tumor.

4. In embodiment 4, the method of any one of embodiments 1 to 3 or 1A, wherein the PARG modulator is a small molecule PARG inhibitor.

5. In embodiment 5, the method of any one of embodiments 1 to 4 or 1A, wherein the PARG modulator is a compound of Formula (I):

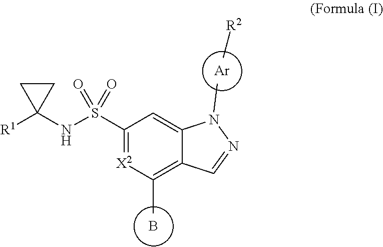

or a pharmaceutically acceptable salt thereof, wherein
R¹ is selected from the group consisting of cyano, C₁₋₂ alkyl, and C₁₋₂ haloalkyl;
Ar is a 5-membered heteroaryl;
X² is CH or CF;
R² is selected from the group consisting of C₁₋₃ alkyl, C₁₋₃ haloalkyl, hydroxyC₁₋₃alkyl, and cyano;
ring B is 5- or 6-membered heterocycloalkyl substituted with R^{a}, R^{b}, and R^{c};
R^{a} is hydrogen, C₁₋₄ alkyl, C₁₋₄ haloalkyl, halo, hydroxy, —C(O)R^{d} (where R^{d} is hydrogen, C₁₋₆ alkyl, or C₁₋₆ haloalkyl); and
R^{b} and R^{c} are independently selected from hydrogen, C₁₋₆ alkyl, hydroxy, C₁₋₆ alkoxy, halo, C₁₋₆ haloalkyl, and C₁₋₆ haloalkoxy.

6. In embodiment 6, the method of embodiment 5, wherein X² is CH.

7. In embodiment 7, the method of embodiment 5 or 6, wherein R¹ is cyano.

8. In embodiment 8, the method of embodiment 5 or 6, wherein R¹ is methyl or ethyl.

9. In embodiment 9, the method of embodiment 5 or 6, wherein R¹ is methyl.

10. In embodiment 10, the method of any one of embodiments 5 to 9, wherein Ar is 1,2,4-thiadiazolyl, or 1,3,4-thiadiazolyl.

11. In embodiment 11, the method of any one of embodiments 5 to 10, wherein Ar is 1,2,4-thiadiazolyl.

12. In embodiment 12, the method of any one of embodiments 5 to 10, wherein Ar is 1,3,4-thiadiazolyl.

13. In embodiment 13, the method of any one of embodiments 5 to 12, wherein R² is attached to the carbon atom of Ar that is meta to the atom of Ar that is attached to the nitrogen atom of the remainder of the molecule.

14. In embodiment 14, the method of any one of embodiments 5 to 13, wherein R² is methyl, ethyl, difluoromethyl, trifluoromethyl, cyano.

15. In embodiment 15, the method of any one of embodiments 5 to 14, wherein R² is difluoromethyl.

16. In embodiment 16, the method of any one of embodiments 5 to 15, wherein ring B is morpholinyl, 1,1-dioxothiomorpholinyl, pyrrolidinyl, piperidinyl, 6-oxo-1,6-dihydropyridinyl, or piperazinyl.

17. In embodiment 17, the method of any one of embodiments 5 to 16, or a pharmaceutically acceptable salt thereof, wherein ring B is piperazinyl.

18. In embodiment 18, the method of any one of embodiments 5 to 17, wherein R^{a} is hydrogen, C₁₋₄ alkyl, C₁₋₄ haloalkyl, halo, hydroxy, —C(O)R^{d} (where R^{d} is hydrogen, C₁₋₆ alkyl, or C₁₋₆ haloalkyl); and R^{b} and R^{c} are independently selected from hydrogen, C₁₋₆ alkyl, hydroxy, C₁-6 alkoxy, halo, C₁₋₆ haloalkyl, and C₁₋₆ haloalkoxy.

19. In embodiment 19, the method of any one of embodiments 5 to 18, wherein R^{a} is hydrogen, C₁₋₄ alkyl, C₁₋₄ haloalkyl, —C(O)R^{d} (where R^{d} is C₁₋₆ alkyl, or C₁₋₆ haloalkyl); and R^{b} and R^{c} are independently selected from hydrogen, C₁₋₆ alkyl, and C₁₋₆ haloalkoxy.

20. In embodiment 20, the method of any one of embodiments 5 to 19, wherein R^{a} is —C(O)R^{d} where R^{d} is C₁₋₆ alkyl; and R^{b} and R^{c} are independently selected from hydrogen, C₁₋₆ alkyl, and C₁₋₆ haloalkoxy.

21. In embodiment 21, the method of any one of embodiments 5 to 19, wherein R^{a} is hydrogen C₁₋₄ alkyl, and C₁₋₄ haloalkyl; and R^{b} and R^{c} are independently selected from hydrogen, and C₁₋₆ alkyl.

22. In embodiment 22, the method of any one of embodiments 5 to 21, wherein the PARG modulator is a compound having the formula:

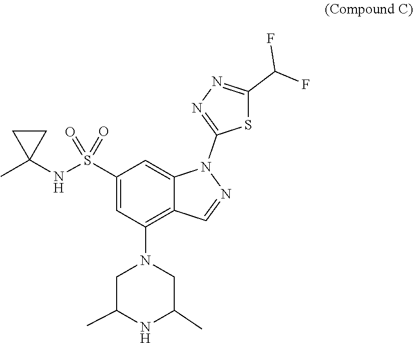 [SMILES: CC1CN(c2cc(S(=O)(=O)NC3(C)CC3)cc3c2cnn3-c2nnc(C(F)F)s2)CC(C)N1]

or a pharmaceutically acceptable salt thereof.

23. In embodiment 23, the method of embodiment 22, wherein the compound is in a free-base form.

24. In embodiment 24, the method of any one of embodiments 5 to 21, wherein the PARG modulator is a compound having the formula:

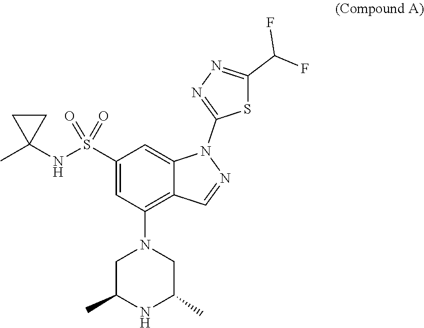 [SMILES: C[C@H]1CN(c2cc(S(=O)(=O)NC3(C)CC3)cc3c2cnn3-c2nnc(C(F)F)s2)C[C@H](C)N1]

or a pharmaceutically acceptable salt thereof.

25. In embodiment 25, the method of embodiment 24, wherein the compound is in a free-base form.

26. In embodiment 26, the method of any one of embodiments 5 to 21, wherein the PARG modulator is selected from the group consisting of:

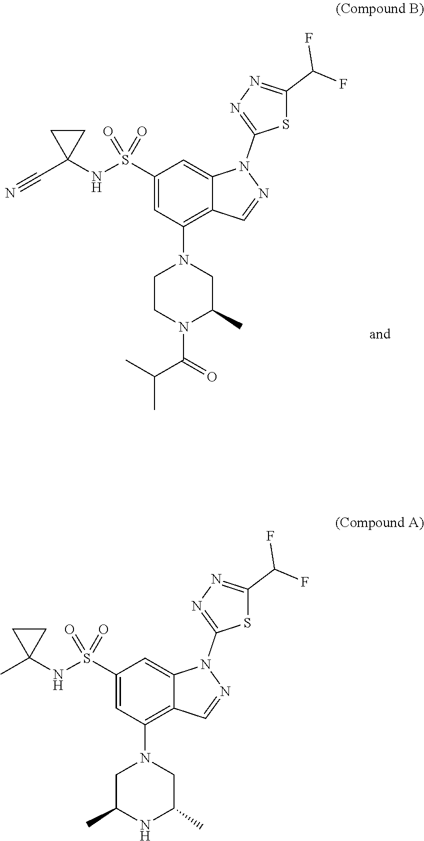 [SMILES: CC(C)C(=O)N1CCN(c2cc(S(=O)(=O)NC3(C#N)CC3)cc3c2cnn3-c2nnc(C(F)F)s2)C[C@H]1C ; C[C@H]1CN(c2cc(S(=O)(=O)NC3(C)CC3)cc3c2cnn3-c2nnc(C(F)F)s2)C[C@H](C)N1]

or a pharmaceutically acceptable salt thereof.

27. In embodiment 27, the method of any one of embodiments 5 to 21, wherein the PARG modulator is a compound having the formula:

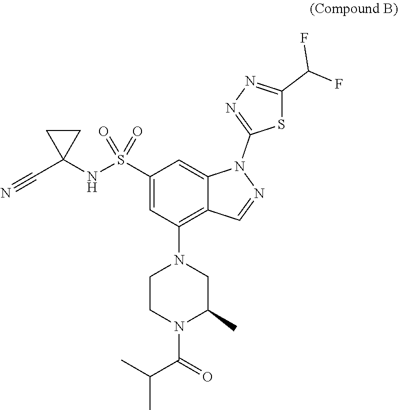 [SMILES: CC(C)C(=O)N1CCN(c2cc(S(=O)(=O)NC3(C#N)CC3)cc3c2cnn3-c2nnc(C(F)F)s2)C[C@H]1C]

or a pharmaceutically acceptable salt thereof.

28. In embodiment 28, the method of embodiment 27, wherein the compound is in a free-base form.

29. In embodiment 29, the method of any one of embodiments 1 to 28, wherein selecting a patient having a breast cancer tumor further comprises obtaining a biological sample from the breast cancer tumor, or obtaining information about the biological sample of the breast cancer tumor.

30. In embodiment 30, the method of embodiment 29, further comprising assessing the biological sample, or evaluating the information about the biological sample, to determine whether the breast cancer tumor comprises:
(a) homologous recombination deficiency (HRD); and
(b) is ER+.

31. In embodiment 31, the method of embodiment 30, further comprising assessing the biological sample, or evaluating the information about the biological sample, to determine whether the breast cancer tumor is HER2-tumor.

32. In embodiment 32, the method of embodiment 30 or 31, further comprising the biological sample, or evaluating the information about the biological sample, to determine whether the breast cancer tumor is PR+ tumor.

33. In embodiment 33, the method of any one of embodiments 29 to 32, further comprising obtaining multiple biological samples, or obtaining information about multiple biological samples, of the breast cancer tumor; wherein the multiple biological samples are taken at separate points of time; and assessing the multiple biological samples, or evaluating the information about multiple biological samples to determine whether the breast cancer tumor:
(a) comprises homologous recombination deficiency (HRD); and
(b) is ER+.

34. In embodiment 34, the method of embodiment 33, further comprising obtaining multiple biological samples, or obtaining information about multiple biological samples, of the breast cancer tumor; wherein the multiple biological samples are taken at separate points of time; and assessing the multiple biological samples, or evaluating the information about multiple biological samples to determine whether the breast cancer tumor is HER2− tumor.

35. In embodiment 35, the method of embodiment 33 or 34, further comprising obtaining multiple biological samples, or obtaining information about multiple biological samples, of the breast cancer tumor; wherein the multiple biological samples are taken at separate points of time; and assessing the multiple biological samples, or evaluating the information about multiple biological samples to determine whether the breast cancer tumor is PR+ tumor.

36. In embodiment 36, the method of any one of embodiments 29 to 35, wherein the biological sample is a biopsy

37. In embodiment 37, the method of embodiment 36, wherein the biopsy is a tumor biopsy, or a liquid biopsy.

38. In embodiment 38, the method of any one of embodiments 29 to 37, wherein the biological sample is a tumor biopsy obtained by core needle biopsy.

39. In embodiment 39, the method of any one of embodiments 29 to 37, wherein the biological sample is a tumor biopsy prepared in the form of tissue sections.

40. In embodiment 40, the method of embodiment 39, wherein the tissue sections are fixed and paraffin-embedded.

41. In embodiment 41, the method of any one of embodiments 29 to 40, wherein the biological sample is frozen.

42. In embodiment 42, the method of any one of embodiments 29 to 41, wherein the biological sample is prepared in the form of isolated cells, lysed cells, homogenate, cell fraction, or a combination thereof.

43. In embodiment 43, the method of embodiment 42, wherein the biological sample is cultured cells.

44. In embodiment 44, the method of any one of embodiments 1 to 43, wherein the homologous recombination deficiency (HRD) is characterized by HRD score, HRDetect, single base substitution signature 3 (SBS3), or indel signature 6 (ID6).

45. In embodiment 45, the method of any one of embodiments 30 to 44, wherein assessing the biological sample or evaluating the information about the biological sample comprises identifying homologous recombination deficiency (HRD) in DNA extracted from a tumor sample.

46. In embodiment 46, the method of any one of embodiments 30 to 45, wherein assessing the biological sample or evaluating the information about the biological sample comprises identifying homologous recombination deficiency (HRD) in a circulating tumor DNA.

47. In embodiment 47, the method of any one of embodiments 29 to 46, wherein assessing the biological sample, or evaluating the information about the biological sample comprises determining whether the breast cancer tumor is ER+ tumor.

48. In embodiment 48, the method of any one of embodiments 29 to 47, wherein assessing the biological sample, or evaluating the information about the biological sample comprises determining whether the breast cancer tumor is HER2− tumor.

49. In embodiment 49, the method of any one of embodiments 29 to 47, wherein assessing the biological sample, or evaluating the information about the biological sample comprises determining whether the breast cancer tumor is PR+ tumor.

50. In embodiment 50, the method of any one of embodiments 1 to 49, wherein the PARG modulator is administered to the patient as a monotherapy.

51. In embodiment 45, the method of any one embodiments 1 to 50, further comprising administering at least one additional therapeutic agent.

### Further Non-Limiting Exemplary Embodiments

In further embodiments 1 to 62 below, the present disclosure includes:

1. In embodiment 1, provided is a method of treating breast cancer in a patient in need thereof, the method comprising:
(a) selecting a patient having a breast cancer tumor wherein the tumor comprises homologous recombination deficiency (HRD) and is estrogen receptor (ER) positive (ER+); and
(b) administering to the patient in need thereof, a therapeutically effective amount of a Poly(ADP-ribose) glycohydrolase (PARG) modulator.

1A. In embodiment 1A, provided is a Poly(ADP-ribose) glycohydrolase (PARG) modulator for use in a method of treating breast cancer in a patient in need thereof, wherein the patient has a breast cancer tumor and the tumor comprises homologous recombination deficiency (HRD) and is estrogen receptor (ER) positive (ER+), the method comprising administering a therapeutically effective amount of the PARG modulator to the patient in need thereof.

1B. In embodiment 1B, provided is a Poly(ADP-ribose) glycohydrolase (PARG) modulator for use in a method of treating breast cancer, said method comprising:
(a) selecting a patient having a breast cancer tumor wherein the tumor comprises homologous recombination deficiency (HRD) and is estrogen receptor (ER) positive (ER+); and
(b) administering to the patient in need thereof, a therapeutically effective amount of the Poly(ADP-ribose) glycohydrolase (PARG) modulator.

1C. In embodiment 1C, provided is the use of a Poly(ADP-ribose) glycohydrolase (PARG) modulator in the manufacture of a medicament for treating breast cancer in a patient in need thereof, wherein the breast cancer in the patient is a tumor and the tumor comprises homologous recombination deficiency (HRD) and is estrogen receptor (ER) positive (ER+).

2. In embodiment 2, the method of embodiment 1, the PARG modulator for use of embodiments 1A or 1B, or the use of embodiment 1C, wherein the breast cancer tumor is a human epidermal growth factor receptor-2 (HER2) negative (HER2−) tumor.

3. In embodiment 3, the method, PARG modulator for use, or use of any one of embodiments 1, 1A, 1B, IC or 2, wherein the breast cancer tumor is a progesterone receptor (PR) positive (PR+) tumor.

4. In embodiment 4, the method, PARG modulator for use, or use of any one of embodiments 1, 1A, 1B, IC, 2 or 3, wherein the PARG modulator is a small molecule PARG inhibitor.

5. In embodiment 5, the method, PARG modulator for use, or use of any one of embodiments 1, 1A, 1B, IC, or 2 to 4, wherein the PARG modulator is a compound of Formula (I):

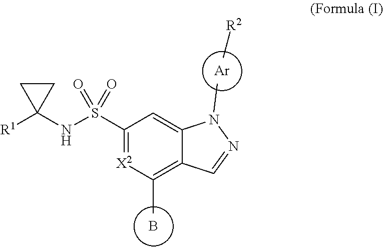

or a pharmaceutically acceptable salt thereof, wherein
R¹ is selected from the group consisting of cyano, C₁₋₂ alkyl, and C₁₋₂ haloalkyl;
Ar is a 5-membered heteroaryl;
X² is CH or CF;
R² is selected from the group consisting of C₁₋₃ alkyl, C₁₋₃ haloalkyl, hydroxyC₁₋₃alkyl, and cyano;
ring B is 5- or 6-membered heterocycloalkyl substituted with R^{a}, R^{b}, and R^{c};
R^{a} is hydrogen, C₁₋₄ alkyl, C₁₋₄ haloalkyl, halo, hydroxy, —C(O)R^{d} (where R^{d} is hydrogen, C₁₋₆ alkyl, or C₁₋₆ haloalkyl); and
R^{b} and R^{c} are independently selected from hydrogen, C₁₋₆ alkyl, hydroxy, C₁₋₆ alkoxy, halo, C₁₋₆ haloalkyl, and C₁₋₆ haloalkoxy.

6. In embodiment 6, the method, PARG modulator for use, or use of embodiment 5, wherein the PARG modulator is a compound of Formula (I) or a pharmaceutically acceptable salt thereof wherein X² is CH.

7. In embodiment 7, the method, PARG modulator for use, or use of embodiment 5 or 6, wherein the PARG modulator is a compound of Formula (I) or a pharmaceutically acceptable salt thereof wherein R¹ is cyano.

8. In embodiment 8, the method, PARG modulator for use, or use of embodiment 5 or 6, wherein the PARG modulator is a compound of Formula (I) or a pharmaceutically acceptable salt thereof wherein R¹ is methyl or ethyl.

9. In embodiment 9, the method, PARG modulator for use, or use of embodiment 5 or 6, wherein the PARG modulator is a compound of Formula (I) or a pharmaceutically acceptable salt thereof wherein R¹ is methyl.

10. In embodiment 10, the method, PARG modulator for use, or use of any one of embodiments 5 to 9, wherein the PARG modulator is a compound of Formula (I) or a pharmaceutically acceptable salt thereof, wherein Ar is 1,2,4-thiadiazolyl, or 1,3,4-thiadiazolyl.

11. In embodiment 11, the method, PARG modulator for use, or use of any one of embodiments 5 to 10, wherein the PARG modulator is a compound of Formula (I) or a pharmaceutically acceptable salt thereof, wherein Ar is 1,2,4-thiadiazolyl.

12. In embodiment 12, the method, PARG modulator for use, or use of any one of embodiments 5 to 10, wherein the PARG modulator is a compound of Formula (I) or a pharmaceutically acceptable salt thereof, wherein Ar is 1,3,4-thiadiazolyl.

13. In embodiment 13, the method, PARG modulator for use, or use of any one of embodiments 5 to 12, wherein the PARG modulator is a compound of Formula (I) or a pharmaceutically acceptable salt thereof, wherein R² is attached to the carbon atom of Ar that is meta to the atom of Ar that is attached to the nitrogen atom of the remainder of the molecule.

14. In embodiment 14, the method, PARG modulator for use, or use of any one of embodiments 5 to 13, wherein the PARG modulator is a compound of Formula (I) or a pharmaceutically acceptable salt thereof, wherein R² is methyl, ethyl, difluoromethyl, trifluoromethyl, cyano.

15. In embodiment 15, the method, PARG modulator for use, or use of any one of embodiments 5 to 14, wherein the PARG modulator is a compound of Formula (I) or a pharmaceutically acceptable salt thereof, wherein R² is difluoromethyl.

16. In embodiment 16, the method, PARG modulator for use, or use of any one of embodiments 5 to 15, wherein the PARG modulator is a compound of Formula (I) or a pharmaceutically acceptable salt thereof, wherein ring B is morpholinyl, 1,1-dioxothiomorpholinyl, pyrrolidinyl, piperidinyl, 6-oxo-1,6-dihydropyridinyl, or piperazinyl.

17. In embodiment 17, the method, PARG modulator for use, or use of any one of embodiments 5 to 16, wherein the PARG modulator is a compound of Formula (I) or a pharmaceutically acceptable salt thereof, wherein ring B is piperazinyl.

18. In embodiment 18, the method, PARG modulator for use, or use of any one of embodiments 5 to 17, wherein the PARG modulator is a compound of Formula (I) or a pharmaceutically acceptable salt thereof, wherein R^{a} is hydrogen, C₁₋₄ alkyl, C₁₋₄ haloalkyl, halo, hydroxy, —C(O)R^{d} (where R^{d} is hydrogen, C₁₋₆ alkyl, or C₁₋₆ haloalkyl); and R^{b} and R^{c} are independently selected from hydrogen, C₁₋₆ alkyl, hydroxy, C₁₋₆ alkoxy, halo, C₁₋₆ haloalkyl, and C₁₋₆ haloalkoxy.

19. In embodiment 19, the method, PARG modulator for use, or use of any one of embodiments 5 to 18, wherein the PARG modulator is a compound of Formula (I) or a pharmaceutically acceptable salt thereof, wherein R^{a} is hydrogen, C₁₋₄ alkyl, C₁₋₄ haloalkyl, —C(O)R^{d} (where R^{d} is C₁₋₆ alkyl, or C₁₋₆ haloalkyl); and R^{b} and R^{c} are independently selected from hydrogen, C₁₋₆ alkyl, and C₁₋₆ haloalkoxy.

20. In embodiment 20, the method, PARG modulator for use, or use of any one of embodiments 5 to 19, wherein the PARG modulator is a compound of Formula (I) or a pharmaceutically acceptable salt thereof, wherein R^{a} is —C(O)R^{d} where R^{d} is C₁₋₆ alkyl; and R^{b} and R^{c} are independently selected from hydrogen, C₁₋₆ alkyl, and C₁₋₆ haloalkoxy.

21. In embodiment 21, the method, PARG modulator for use, or use of any one of embodiments 5 to 19, wherein the PARG modulator is a compound of Formula (I) or a pharmaceutically acceptable salt thereof, wherein R^{a} is hydrogen C₁₋₄ alkyl, and C₁₋₄ haloalkyl; and R^{b} and R^{c} are independently selected from hydrogen, and C₁₋₆ alkyl.

22. In embodiment 22, the method, PARG modulator for use, or use of any one of embodiments 5 to 21, wherein the PARG modulator is a compound having the formula:

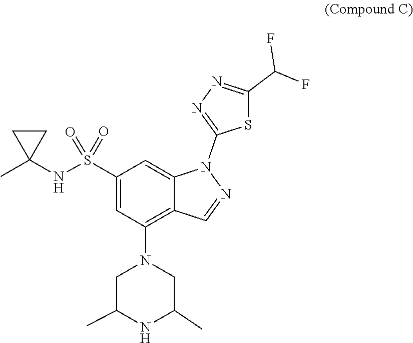 [SMILES: CC1CN(c2cc(S(=O)(=O)NC3(C)CC3)cc3c2cnn3-c2nnc(C(F)F)s2)CC(C)N1]

or a pharmaceutically acceptable salt thereof.

23. In embodiment 23, the method, PARG modulator for use, or use of embodiment 22, wherein the compound is in a free-base form.

24. In embodiment 24, the method, PARG modulator for use, or use of any one of embodiments 5 to 21, wherein the PARG modulator is a compound having the formula:

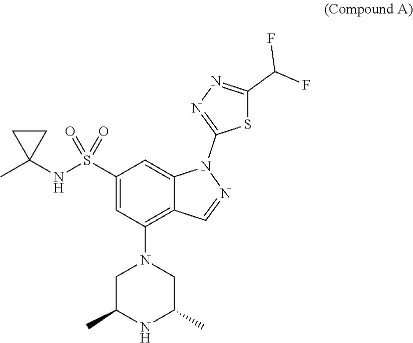 [SMILES: C[C@H]1CN(c2cc(S(=O)(=O)NC3(C)CC3)cc3c2cnn3-c2nnc(C(F)F)s2)C[C@H](C)N1]

or a pharmaceutically acceptable salt thereof.

25. In embodiment 25, the method, PARG modulator for use, or use of embodiment 24, wherein the compound is in a free-base form.

26. In embodiment 26, the method, PARG modulator for use, or use of any one of embodiments 5 to 21, wherein the PARG modulator is selected from the group consisting of:

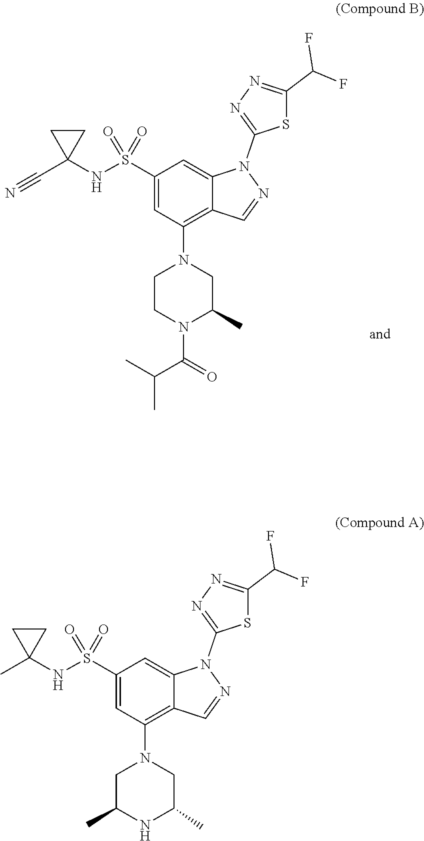 [SMILES: CC(C)C(=O)N1CCN(c2cc(S(=O)(=O)NC3(C#N)CC3)cc3c2cnn3-c2nnc(C(F)F)s2)C[C@H]1C ; C[C@H]1CN(c2cc(S(=O)(=O)NC3(C)CC3)cc3c2cnn3-c2nnc(C(F)F)s2)C[C@H](C)N1]

or a pharmaceutically acceptable salt thereof.

27. In embodiment 27, the method, PARG modulator for use, or use of any one of embodiments 5 to 21, wherein the PARG modulator is a compound having the formula:

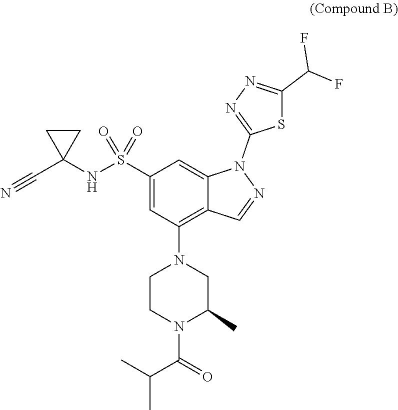 [SMILES: CC(C)C(=O)N1CCN(c2cc(S(=O)(=O)NC3(C#N)CC3)cc3c2cnn3-c2nnc(C(F)F)s2)C[C@H]1C]

or a pharmaceutically acceptable salt thereof.

28. In embodiment 28, the method, PARG modulator for use, or use of embodiment 27, wherein the compound is in a free-base form.

29. In embodiment 29, the method of embodiment 1, PARG modulator for use of embodiment IB, or the method or PARG modulator for use of embodiments 2 to 28 (so far as they depend on the method of embodiment 1 or the PARG modulator for use of embodiment IB), wherein selecting a patient having a breast cancer tumor further comprises obtaining a biological sample from the breast cancer tumor, or obtaining information about a biological sample of the breast cancer tumor.

30. In embodiment 30, the method or the PARG modulator for use of embodiment 29, further comprising assessing the biological sample, or evaluating the information about the biological sample, to determine whether the breast cancer tumor:
(a) comprises homologous recombination deficiency (HRD); and
(b) is ER+.

31. In embodiment 31, the method or the PARG modulator for use of embodiment 30, further comprising assessing the biological sample, or evaluating the information about the biological sample, to determine whether the breast cancer tumor is HER2-tumor.

32. In embodiment 32, the method or the PARG modulator for use of embodiment 30 or 31, further comprising assessing the biological sample, or evaluating the information about the biological sample, to determine whether the breast cancer tumor is PR+ tumor.

33. In embodiment 33, the method or the PARG modulator for use of any one of embodiments 29 to 32, further comprising obtaining multiple biological samples, or obtaining information about multiple biological samples, of the breast cancer tumor; wherein the multiple biological samples are taken at separate points of time; and assessing the multiple biological samples, or evaluating the information about multiple biological samples to determine whether the breast cancer tumor:
(a) comprises homologous recombination deficiency (HRD); and
(b) is ER+.

34. In embodiment 34, the method or the PARG modulator for use of embodiment 33, further comprising obtaining multiple biological samples, or obtaining information about multiple biological samples, of the breast cancer tumor; wherein the multiple biological samples are taken at separate points of time; and assessing the multiple biological samples, or evaluating the information about multiple biological samples to determine whether the breast cancer tumor is HER2-tumor.

35. In embodiment 35, the method or the PARG modulator for use of embodiment 33 or 34, further comprising obtaining multiple biological samples, or obtaining information about multiple biological samples, of the breast cancer tumor; wherein the multiple biological samples are taken at separate points of time; and assessing the multiple biological samples, or evaluating the information about multiple biological samples to determine whether the breast cancer tumor is PR+ tumor.

36. In embodiment 36, the method or the PARG modulator for use of any one of embodiments 29 to 35, wherein the biological sample is a biopsy

37. In embodiment 37, the method or the PARG modulator for use of embodiment 36, wherein the biopsy is a tumor biopsy, or a liquid biopsy.

38. In embodiment 38, the method or the PARG modulator for use of any one embodiments 29 to 37, wherein the biological sample is a tumor biopsy obtained by core needle biopsy.

39. In embodiment 39, the method or the PARG modulator for use of any one of embodiments 29 to 37, wherein the biological sample is a tumor biopsy prepared in the form of tissue sections.

40. In embodiment 40, the method or the PARG modulator for use of embodiment 39, wherein the tissue sections are fixed and paraffin-embedded.

41. In embodiment 41, the method or the PARG modulator for use of any one of embodiments 29 to 40, wherein the biological sample is frozen.

42. In embodiment 42, the method or the PARG modulator for use of any one of embodiments 29 to 41, wherein the biological sample is prepared in the form of isolated cells, lysed cells, homogenate, cell fraction, or a combination thereof.

43. In embodiment 43, the method or the PARG modulator for use of embodiment 42, wherein the biological sample is cultured cells.

44. In embodiment 44, the method, or the PARG modulator for use, or use of any one of embodiments 1 to 43, wherein the homologous recombination deficiency (HRD) is characterized by HRD score, HRDetect, single base substitution signature 3 (SBS3), or indel signature 6 (ID6).

45. In embodiment 45, the method or the PARG modulator for use of any one of embodiments 30 to 44, wherein assessing the biological sample or evaluating the information about the biological sample comprises identifying homologous recombination deficiency (HRD) in DNA extracted from a tumor sample.

46. In embodiment 46, the method or the PARG modulator for use of any one of embodiments 30 to 45, wherein assessing the biological sample or evaluating the information about the biological sample comprises identifying homologous recombination deficiency (HRD) in a circulating tumor DNA.

47. In embodiment 47, the method or the PARG modulator for use of any one of embodiments 29 to 46, wherein assessing the biological sample, or evaluating the information about the biological sample comprises determining whether the breast cancer tumor is ER+ tumor.

48. In embodiment 48, the method or the PARG modulator for use of any one of embodiments 29 to 47, wherein assessing the biological sample, or evaluating the information about the biological sample comprises determining whether the breast cancer tumor is HER2− tumor.

49. In embodiment 49, the method or the PARG modulator for use of any one of embodiments 29 to 47, wherein assessing the biological sample, or evaluating the information about the biological sample comprises determining whether the breast cancer tumor is PR+ tumor.

50. In embodiment 50, the method, the PARG modulator for use, or use of any one of embodiments 1 to 49, wherein the PARG modulator is administered to the patient as a monotherapy.

51. In embodiment 51, the method, the PARG modulator for use, or use of any one embodiments 1 to 50, further comprising administering at least one additional therapeutic agent.

52. In embodiment 52, a method of identifying a breast cancer patient susceptible to treatment with a Poly(ADP-ribose) glycohydrolase (PARG) modulator, the method comprising:
(a) identifying a patient having a breast cancer tumor, and
(b) obtaining information about a biological sample of the breast cancer tumor and evaluating the information about the biological sample to determine whether the breast cancer tumor comprises homologous recombination deficiency (HRD) and is estrogen receptor (ER) positive (ER+).

53. In embodiment 53, the method according to embodiment 52, further comprising evaluating the information about the biological sample to determine whether the breast cancer tumor is a HER2-tumor.

54. In embodiment 54, the method according to embodiment 52 or 53, further comprising evaluating the information about the biological sample to determine whether the breast cancer tumor is a PR+ tumor.

55. In embodiment 55, the method according to any one of embodiments 52 to 54, comprising obtaining information about multiple biological samples of the breast cancer tumor, wherein the multiple biological samples are taken at separate points of time, and evaluating the information about the multiple biological samples to determine whether the breast cancer tumor:
(a) comprises homologous recombination deficiency (HRD); and
(b) is ER+

56. In embodiment 56, the method according to embodiment 55, comprising obtaining information about multiple biological samples of the breast cancer tumor, wherein the multiple biological samples are taken at separate points of time, and evaluating the information about the multiple biological samples to determine whether the breast cancer tumor is HER2-tumor.

57. In embodiment 57, the method according to embodiment 55 or 56, comprising obtaining information about multiple biological samples of the breast cancer tumor, wherein the multiple biological samples are taken at separate points of time, and evaluating the information about the multiple biological samples to determine whether the breast cancer tumor is is PR+ tumor.

58. In embodiment 58, the method according to any one of embodiments 52 to 58, wherein the biological sample is a biological sample as defined in any one of embodiments 36 to 43.

59. In embodiment 59, the method according to any one of embodiments 52 to 58, wherein the homologous recombination deficiency (HRD) is characterized by HRD score, HRDetect, single base substitution signature 3 (SBS3), or indel signature 6 (ID6).

60. In embodiment 60, the method according to any one of embodiments 52 to 59, wherein evaluating the information about the biological sample comprises identifying homologous recombination deficiency (HRD) in DNA extracted from a tumor sample.

61. In embodiment 61, the method according to any one of embodiments 52 to 60, wherein evaluating the information about the biological sample comprises identifying homologous recombination deficiency (HRD) in a circulating tumor DNA.

62. In embodiment 62, the method according to any one of embodiments 52 to 61, wherein the PARG modulator is a compound as defined in any one of embodiments 5 to 28.

### EXAMPLES

The Examples in this specification are not intended to, and should not be used to, limit the disclosure; they are provided only to illustrate the disclosure.

### Example 1: Synthesis of Compound A

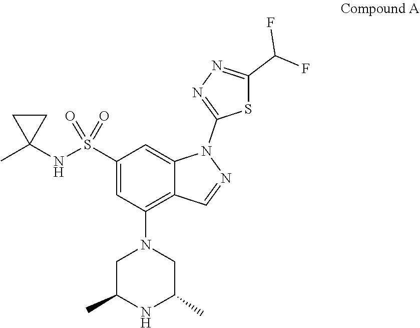 [SMILES: C[C@H]1CN(c2cc(S(=O)(=O)NC3(C)CC3)cc3c2cnn3-c2nnc(C(F)F)s2)C[C@H](C)N1]

### Step 1: Preparation of 2,6-difluoro-4-iodobenzaldehyde

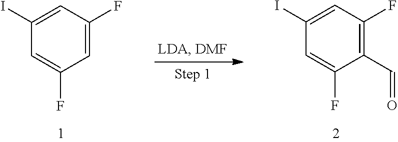 [SMILES: Fc1cc(F)cc(I)c1 ; O=Cc1c(F)cc(I)cc1F]

To a stirred solution of 1,3-difluoro-5-iodobenzene (Compound 1) (50 g, 208.3 mmol, Oakwood, CAS 2265-91-0, catalogue #024556) in THF (500 mL) was added LDA (80 mL, 625.0 mmol) and DMF (48.3 mL, 625 mmol) at −78° C. and stirred at −78° C. for 2 h. After complete consumption of starting material, the reaction mixture was diluted with water (500 mL) and extracted with EtOAc (2×300 mL), washed with brine solution (200 mL), dried over anhydrous sodium sulphate, filtered and concentrated under reduced pressure to get crude product as an oil. The crude material was purified by column chromatography using silica gel (100-200) and eluted with 20% EtOAc/Hexane as a gradient. The product was eluted with a gradient of 30% EtOAc/Hexane. The purified fractions were concentrated under reduced pressure to afford 2,6-difluoro-4-iodobenzaldehyde (Compound 2) (23 g) as a solid. ¹H NMR (500 MHz, CHLOROFORM-d) δ:10.29 (s, 1H), 7.37-7.46 (m, 2H).

### Step 2: Preparation of 4-fluoro-6-iodo-1H-indazole

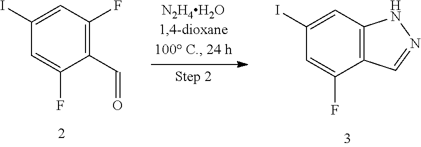 [SMILES: O=Cc1c(F)cc(I)cc1F ; Fc1cc(I)cc2[nH]ncc12]

To a stirred solution of 2,6-difluoro-4-iodobenzaldehyde (Compound 2) (5 g, 18.6 mmol) in 1,4 dioxane (110 mL) was added hydrazine hydrate (18.6 mL, 373.1 mmol) at RT and stirred at 100° C. for 24 h. The reaction mixture was concentrated under reduced pressure and added ice cold water (100 mL) then stirred for 30 min. The solid was precipitated out which was filtered, washed with water (100 mL), n-pentane (50 mL), and dried under vacuum to afford product 4-fluoro-6-iodo-1H-indazole (Compound 3) (2.3 g) as a solid. MS ESI calculated for C₇H₄FIN₂ [M+H]⁺262.94. found 262.99. ¹H NMR (CDCl₃, 400 MHz): 10.12 (s, 1H), 8.10 (s, 1H), 7.70 (s, 1H), 7.15 (dd, J=9 Hz, 1H).

### Step 3: Preparation of 2-(difluoromethyl)-5-(4-fluoro-6-iodo-1H-indazol-1-yl)-1,3,4-thiadiazole

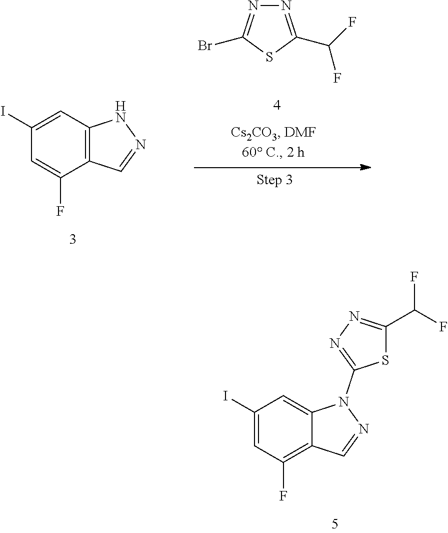 [SMILES: FC(F)c1nnc(Br)s1 ; Fc1cc(I)cc2[nH]ncc12 ; Fc1cc(I)cc2c1cnn2-c1nnc(C(F)F)s1]

To a stirred solution of 4-fluoro-6-iodo-1H-indazole (Compound 3) (5 g, 19.0 mmol) in DMF (50 mL) was added cesium carbonate (18.6 g, 57.24 mmol) and 2-bromo-5-(difluoromethyl)-1,3,4-thiadiazole (Compound 4) (3.8 g, 18.1 mmol, Enamine Stock Building Blocks, CAS 1340313-49-6, catalogue #EN300-108825) and stirred at 60° C. for 2 h. The progress of the reaction was monitored by TLC. The reaction mixture was quenched with ice cold water (50 mL) and stirred for 30 min. The solid that precipitated out was filtered, washed with water (100 mL) followed by n-pentane (100 mL), and dried under vacuum to afford 2-(difluoromethyl)-5-(4-fluoro-6-iodo-1H-indazol-1-yl)-1,3,4-thiadiazole (Compound 5) (4.2 g) as a solid. MS ESI calculated for C₁₀H₄F₃IN₄S [M+H]⁺ 396.92. found 396.91. ¹H NMR (CDCl₃, 500 MHz): 8.87 (s, 1H), 8.29 (s, 1H), 7.40 (dd, J=17 Hz, 1H), 7.0 (t, J=53.5 Hz, 1H).

### Step 4: Preparation of S-(1-(5-(difluoromethyl)-1,3,4-thiadiazol-2-yl)-4-fluoro-1H-indazol-6-yl)benzothioate

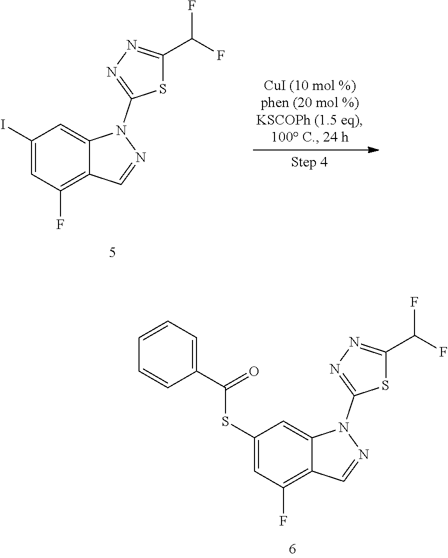 [SMILES: Fc1cc(I)cc2c1cnn2-c1nnc(C(F)F)s1 ; O=C(Sc1cc(F)c2cnn(-c3nnc(C(F)F)s3)c2c1)c1ccccc1]

To a stirred solution of 2-(difluoromethyl)-5-(4-fluoro-6-iodo-1H-indazol-1-yl)-1,3,4-thiadiazole (Compound 5) (100 mg, 0.25 mmol) in toluene (1 mL) degassed for 5 min, was added CuI (5 mg, 0.025 mmol), 1,10-phenanthroline (phen) (11 mg, 0.05 mmol), and potassium thiobenzoate (67 mg, 0.378 mmol) at RT. The system was stirred at 100° C. for 16 h. The progress of the reaction was monitored by LCMS. The crude compound was purified by column chromatography using silica gel (100-200) and eluted with 10% EtOAc/Hexane as a gradient. The purified fractions were collected and concentrated under reduced pressure to afford S-(1-(5-(difluoromethyl)-1,3,4-thiadiazol-2-yl)-4-fluoro-1H-indazol-6-yl)benzothioate (Compound 6) (55 mg) as a solid. MS ESI calculated for C₁₇H₉F₃N₄OS₂ [M+H]⁺407.02. found 407.01. ¹H NMR (CDCl₃, 400 MHz): 8.68 (s, 1H), 8.39 (s, 1H), 8.03 (d, J=7.6 Hz, 2H), 7.64 (t, J=7.2 Hz, 1H), 7.53 (t, J=7.6 Hz, 2H), 7.27 (s, 1H), 6.99 (t, J=53.2 Hz, 1H).

### Step 5: Preparation of N-(1-cyanocyclopropyl)-1-(5-(difluoromethyl)-1,3,4-thiadiazol-2-yl)-4-fluoro-1H-indazole-6-sulfonamide

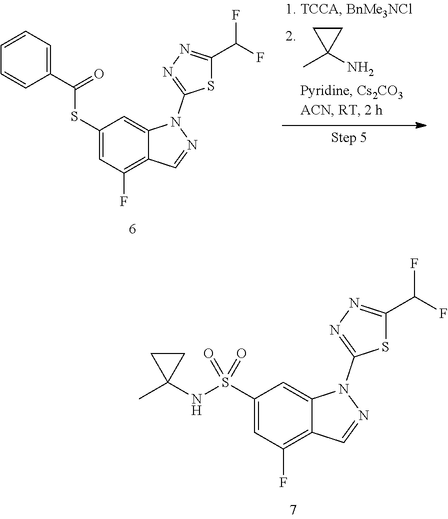

To a stirred solution of S-(1-(5-(difluoromethyl)-1,3,4-thiadiazol-2-yl)-4-fluoro-1H-indazol-6-yl)benzothioate (Compound 6) (500 mg, 1.23 mmol) in acetonitrile (10 mL) at 0° C. were added a solution of BnMe₃NCI (682 mg, 3.69 mmol), and TCCA (trichloroisocyanuricacid) (370 mg 1.59 mmol) in acetonitrile (40 mL). The reaction mixture was stirred for 20 min. Then a solution of 1-methyl cyclopropane-1-amine (1.71 g, 7.38 mmol, Combi-Blocks, CAS 22936-83-0, catalogue #QH-3696) in pyridine (2.5 mL) and cesium carbonate (198 mg, 0.61 mmol) were added to the reaction mixture at 0° C. and stirred at RT for 2 h. The progress of the reaction was monitored by LCMS. LCMS showed complete consumption of starting material (S-(1-(5-(difluoromethyl)-1,3,4-thiadiazol-2-yl)-4-fluoro-1H-indazol-6-yl)benzothioate). The reaction mixture was diluted with water (50 mL) and extracted with EtOAc (2×30 mL). The combined organic layer was washed with brine solution (20 mL), dried over anhydrous sodium sulphate, filtered and concentrated under reduced pressure to obtain crude product. The crude product was purified by column chromatography using silica gel (100-200) and eluted with 5 to 50% EtOAc/hexane as a gradient. The product was eluted at 20% EtOAc/hexane. The purified fractions were collected and concentrated under reduced pressure to afford 1-(5-(difluoromethyl)-1,3,4-thiadiazol-2-yl)-4-fluoro-N-(1-methylcyclopropyl)-1H-indazole-6-sulfonamide (Compound 7) (90 mg) as a solid. MS ESI calculated for C₁₄H₉F₃N₆O₂S₂ [M+H]⁺404.04. found 404.18. ¹H NMR (CDCl₃, 400 MHz): 9.00 (s, 1H), 8.80 (s, 1H), 8.54 (s, 1H), 7.63 (t, J=48.8 Hz, 2H), 1.10 (s, 3H), 0.65 (s, 2H), 0.44 (s, 2H).

### Step 6: Preparation of tert-butyl(2S,6S)-4-(1-(5-(difluoromethyl)-1,3,4-thiadiazol-2-yl)-6-(N-(1-methylcyclopropyl) sulfamoyl)-1H-indazol-4-yl)-2,6-dimethylpiperazine-1-carboxylate

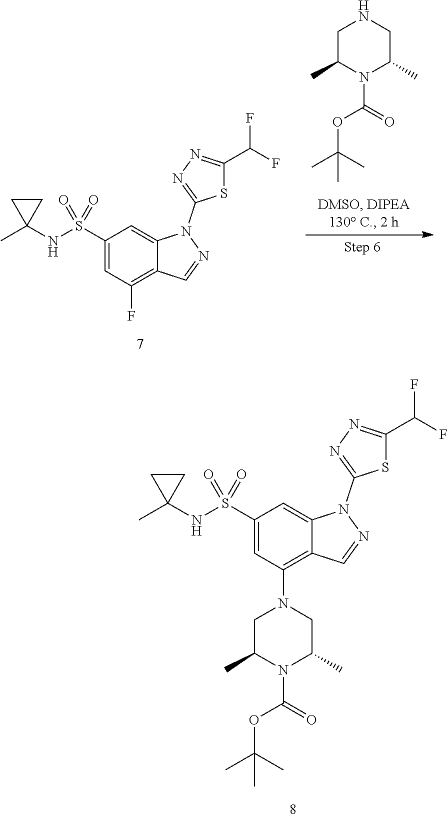

To a stirred solution of 1-(5-(difluoromethyl)-1,3,4-thiadiazol-2-yl)-4-fluoro-N-(1-methylcyclopropyl)-1H-indazole-6-sulfonamide (Compound 7) (80 mg, 0.19 mmol) in DMSO (dimethyl sulfoxide) (2 mL) were added tert-butyl(2S,6S)-2,6-dimethylpiperazine-1-carboxylate (85 mg, 0.39 mmol, BLD, CAS 574007-66-2, catalogue #BD233798) and DIPEA (N,N-diisopropyl ethylamine) (0.1 mL, 0.59 mmol) and reaction mixture was stirred at 130° C. for 2 h. The reaction mixture was quenched with ice cold water (20 mL) and stirred for 30 min. The obtained solid was filtered, washed with water (10 mL), dried under vacuum and purified by column chromatography over silica gel (100-200) and eluted with 50% EtOAc/hexane as a gradient, purified fractions concentrated under reduced pressure to afford tert-butyl(2S,6S)-4-(1-(5-(difluoromethyl)-1,3,4-thiadiazol-2-yl)-6-(N-(1-methylcyclopropyl) sulfamoyl)-1H-indazol-4-yl)-2,6-dimethylpiperazine-1-carboxylate (Compound 8) (110 mg, yield: 92%) as a solid. MS ESI calculated for C₂₅H₃₃F₂N₇O₄S₂ [M+H]⁺598.20, found 598.26.

### Step 7: Preparation of 1-(5-(difluoromethyl)-1,3,4-thiadiazol-2-yl)-4-((3S,5S)-3,5-dimethylpiperazin-1-yl)-N-(1-methylcyclopropyl)-1H-indazole-6-sulfonamide

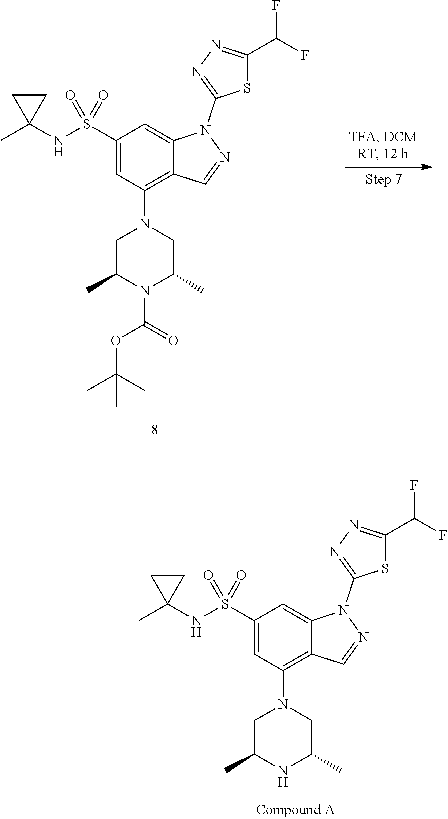 [SMILES: C[C@H]1CN(c2cc(S(=O)(=O)NC3(C)CC3)cc3c2cnn3-c2nnc(C(F)F)s2)C[C@H](C)N1C(=O)OC(C)(C)C ; C[C@H]1CN(c2cc(S(=O)(=O)NC3(C)CC3)cc3c2cnn3-c2nnc(C(F)F)s2)C[C@H](C)N1]

To a stirred solution of tert-butyl(2S,6S)-4-(1-(5-(difluoromethyl)-1,3,4-thiadiazol-2-yl)-6-(N-(1-methylcyclopropyl) sulfamoyl)-1H-indazol-4-yl)-2,6-dimethyl piperazine-1-carboxylate (Compound 8) (100 mg, 0.16 mmol) in DCM (3 mL) was added trifluoroacetic acid (0.07 mL, 0.98 mmol) at 0° C. and reaction mixture was stirred at RT for 12 h. The reaction mixture was concentrated under reduced pressure, purified by Prep HPLC purification (Prep HPLC conditions: MOBILE PHASE—10 mM ammonium bicarbonate in H₂O: MeCN COLUMN—Inertsil ODS (20×250) mm 5u Flow-18 ml/min GRADIENT METHOD—0/50, 9.5/82, 9.55/99, 11.5/99, 11.55/50, 14.5/50, SOLUBILITY: CAN, Fraction Volume: 100 mL) to afford 1-(5-(difluoromethyl)-1,3,4-thiadiazol-2-yl)-4-((3S,5S)-3,5-dimethylpiperazin-1-yl)-N-(1-methylcyclopropyl)-1H-indazole-6-sulfonamide (Compound A) (18 mg, yield: 21%) as a solid. MS ESI calculated for C₂₀H₂₅F₂N₇O₂S₂ [M+H]⁺498.15, found 498.34. ¹H NMR (DMSO-d₆, 400 MHz): δ (ppm) 8.75 (s, 1H), 8.40 (s, 1H), 8.31 (s, 1H), 7.59 (t, J=52.8 Hz, 1H), 7.10 (d, J=1.0 Hz, 1H), 3.37 (br dd, J=11.5, 2.9 Hz, 2H), 3.22-3.30 (m, 2H), 3.08 (br dd, J=11.7, 6.1 Hz, 2H), 1.17 (d, J=6.4 Hz, 6H), 1.08 (s, 3H), 0.58-0.76 (m, 2H), 0.29-0.49 (m, 2H).

### Example 2: Synthesis of Compound B

Compound B can be synthesized according to procedure described in WO2021/055744.

### Example 3: Inhibition of PARG Enzymatic Assay (TR-FRET)

### Enzymatic EC₅₀ Assay

PARG enzyme was incubated with compound or vehicle (DMSO) and the biotinylated-PARylated PARP-1 substrate in a microtiter plate. After adding detection antibody and streptavidin-europium, and then incubating, the plate was read for fluorescence intensity. The low control (DMSO) with low fluorescence intensity represents no inhibition of enzymatic activity, while the high control (no enzyme) with high fluorescence intensity represents full inhibition of enzymatic activity.

### Materials:

Enzyme:
   PARG
      hPARG: 250 μM, 1-976, His-tagged, Proteos, 2.0 mg/mL (17.9 μM)
      Substrate: 30 nM
      Test Compound/Enzyme Pre-incubation time: 1 hr
      Enzyme/Substrate Reaction time: 10 minutes
Substrate: hPARP1, His6-TEV tagged, 1.2 mg/ml (10.3 μM)
Detection Antibody: anti-His monoclonal antibody-ULight, Perkin Elmer catalog #TRF0134-M
Streptavidin-Europium: Perkin Elmer catalog #AD0062
Assay Buffer: 50 mM Tris-HCL pH 7.4, 50 mM KCL, 3 mM EDTA, 0.4 mM EGTA, 1 mM DTT, 0.01% Tween 20, 0.01% BSA
Temperature: 23° C.
Total reaction volume: 20 μL
Controls:
   0% inhibition: DMSO
   100% inhibiton: No enzyme

### Enzyme Reaction and Detection:

1. Transfer 200 nL of 100×compound or DMSO to the appropriate wells of a 384 well white polystyrene microtitre plate (Corning Catalog #3574).
2. Transfer 10 μL of 2×final concentration of enzyme in assay buffer or assay buffer alone to the appropriate wells.
3. Centrifuge the plate at 1000 rpm for 30 seconds.
4. Incubate the plate at room temperature for 1 hour.
5. Transfer 10 μL of 2×substrate in assay buffer to all test wells.
6. Incubate the plate at room temperature for 10 minutes
7. Transfer 10 μL of 3×mixture of 42 nM detection antibody and 2.25 nM streptavidin-europium in 50 mM Tris-HCL pH 7.4 to all test wells.
8. Incubate the plate at room temperature for 1 hour.
9. Read the plate on a plate reader (Envision)
   Excitation: 317 nM
   Emission: 620 nM
   Emission: 665 nM

### Data Analysis:

EC₅₀ values were calculated in Collaborative Drug Discovery vault (CDD). Curves were fitted by CDD as response (%) vs compound concentration (uM) using a 4-parameter inhibition model using Formula 1.

$$\begin{matrix}Formula 1 & \\ Fit = ( A + ( ( B - A ) / ( 1 + ( ( C / x ) \hat{} D ) ) ) ) & A\end{matrix}$$

$$Res = ( y - fit ) \qquad B$$

The TR-FRET EC₅₀ value for Compound A and Compound B is provided in Table 1, below.

TABLE 1
TR-FRET Assay Results for
Compounds of Formula (I).

| Compound | TR-FRET |
|---|---|
| No. | IC₅₀ (UM) |
| Compound | **** |
| A | |
| Compound | **** |
| B | |

### Example 4: Cellular Antiproliferative Response to Compound a Stratified by HR Mutant Status

### Assay 1

Compound A was tested in a cell line panel. Cell lines were obtained from several different sources such as the American Type Culture Collection, Korean Cell Line Bank and Japanese Collection of Research Bioresources Cell Bank and cultured in the media as recommended by the source cell banks. All cell lines were grown at 37° C. at 5% CO₂ and were passaged once or twice weekly with 0.25% Trypsin-EDTA (Fisher Scientific #25200056) and re-plated in 75 cm² culture flasks (Corning #3814).

The cell lines were plated in 96 well plates (Corning #3904) at density of 1000 cells/well. DMSO dissolved Compound A was then added using the TECAN liquid dispenser to generate a 9-point dose curve with a 3-fold dilution and 10 μM starting, top concentration. After 5 population doublings, the cell lines were treated with 5 μM of Vybrant DyeCycle Green (Life Technologies #V35004), incubated for 60 minutes and imaged using the Incucyte® S3 system to determine the nuclear counts. For all cell lines, the nuclear counts were normalized to the DMSO treated wells and the IC₅₀ was determined using the standard four parametric dose response equation in GraphPad Prism Software.

### Assay 2

Compound A was tested in a cell line panel. Cell lines were grown in RPMI 1640, 10% FBS, 2 mM L-alanyl-L-glutamine, 1 mM Na pyruvate, or a special medium. Cells were seeded into 384-well plates and incubated in a humidified atmosphere of 5% CO2 at 37° C. The following day, Compound A was added by serial dilution in half-log steps from the highest test concentration of 30 μM and assayed over ten concentrations. A time zero untreated cell plate was also generated. At seven days post-seeding, the growth media were replaced, and the plates were re-dosed with the test compound. After a 10-day incubation period, cells were fixed and stained with a fluorescent nuclear dye. Automated fluorescence microscopy was carried out using a Molecular Devices ImageXpress Micro XL high-content imager, and images were collected with a 4× objective. 16-bit TIFF images were acquired and analyzed with MetaXpress 5.1.0.41 software.

Cell proliferation effects of Compound A was measured using the fluorescent intensity of the incorporated nuclear dye as a percent of the control (POC), where POC=(Ix/I0)×100. Ix=nuclear intensity at concentration x and I0=average nuclear intensity of the untreated vehicle wells. Cellular IC50 was calculated using nonlinear regression to a sigmoidal single-site dose response model. Curve fitting, calculations, and report generation was performed using a custom data reduction engine and MathIQ based software (AIM).

### Bioinformatic Analysis on Breast Cancer Cell Line Panel

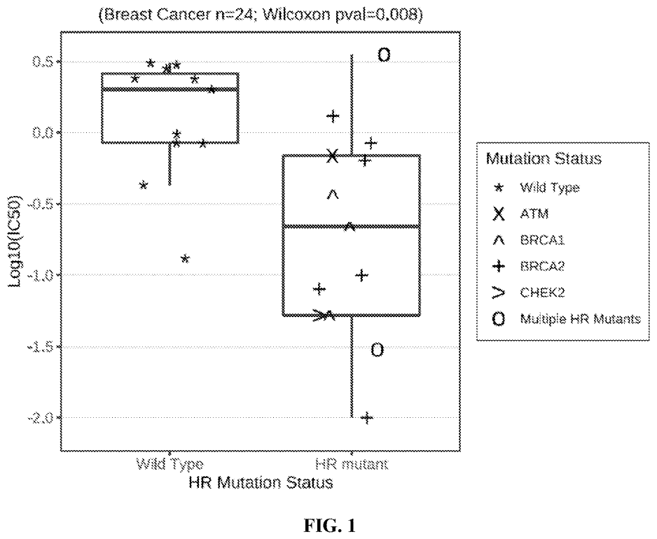

Univariate statistical testing was performed on the panel of 24 breast cancer cell lines. See Table 2 and FIG. 1. All statistical tests were carried out in the Python programming language with the scipy library (Virtanen 2020). A preference in all calculations was made for non-parametric methods given the low sample size and inability to ensure that gaussian approximations would hold. Reported statistics utilize the two-sided Mann-Whitney U Test (Wilcoxon Rank Sum Test). When applicable, multi-test correction was performed with the Benjamini-Hochberg method (Benjamini 2001) for false discovery rate correction.

Analysis was conducted using a set of clinically relevant HRD alterations defined by Foundation Medicine and approved as a companion diagnostic for metastatic castration-resistant prostate cancer (Milbury 2022). This set of fourteen genes (ATM, BRCA1, BRCA2, BARD1, BRIP1, CDK12, CHEK1, CHEK2, FANCL, PALB2, RAD51B, RAD51C, RAD51D, RAD54L) was used such that a cell line with a pathogenic alteration in any of these fourteen genes would be considered HR deficient.

### REFERENCES

Virtanen, Pauli et al. “SciPy 1.0: fundamental algorithms for scientific computing in Python.” Nature methods vol. 17, 3 (2020): 261-272.
Benjamini, Y et al. “Controlling the false discovery rate in behavior genetics research.” Behavioural brain research vol. 125, 1-2 (2001): 279-84.
Milbury, Coren A et al. “Clinical and analytical validation of FoundationOne®CDx, a comprehensive genomic profiling assay for solid tumors.” PloS one vol. 17, 3 e0264138.

TABLE 2

| | | | | | Shape |
|---|---|---|---|---|---|
| | | | | | in |
| | Compound A | Mutation | | Assay | FIG. |
| Cell Line | (Log10 (IC50)) | Status | Mutant | used | 1 |
| SK-BR-3 | −0.882728704 | Wild Type | | Assay 1 | * |
| KPL-1 | −0.36552273 | Wild Type | | Assay 1 | * |
| CAMA-1 | −0.073657553 | Wild Type | | Assay 1 | * |
| MDA-MB- | −0.070581074 | Wild Type | | Assay 1 | * |
| 231 | | | | | |
| BT-549 | −0.007446482 | Wild Type | | Assay 1 | * |
| MDA-MB- | 0.305351369 | Wild Type | | Assay 1 | * |
| 453 | | | | | |
| MCF7 | 0.376576957 | Wild Type | | Assay 1 | * |
| BT-20 | 0.382017043 | Wild Type | | Assay 1 | * |
| MDA-MB- | 0.451786436 | Wild Type | | Assay 1 | * |
| 415 | | | | | |
| Hs 578T | 0.477121255 | Wild Type | | Assay 1 | * |
| T-47D | 0.489958479 | Wild Type | | Assay 1 | * |
| HCC1428 | −2 | HR mutant | BRCA2 | Assay 2 | + |
| HCC1395 | −1.522878745 | HR mutant | BRCA1, | Assay 2 | o |
| | | | BRCA2 | | |
| AU565 | −1.281498311 | HR mutant | CHEK2 | Assay 2 | > |
| MDA-MB- | −1.280807946 | HR mutant | BRCA1 | Assay 2 | \hat{ } |
| 436 | | | | | |
| HCC1569 | −1.096910013 | HR mutant | BRCA2 | Assay 2 | + |
| CAL-85-1 | −1 | HR mutant | BRCA2 | Assay 2 | + |
| HCC1937 | −0.657577319 | HR mutant | BRCA1 | Assay 2 | \hat{ } |
| HCC1954 | −0.431798276 | HR mutant | BRCA1 | Assay 2 | \hat{ } |
| HCC202 | −0.193820026 | HR mutant | BRCA2 | Assay 2 | + |
| EFM-19 | −0.161780778 | HR mutant | ATM | Assay 2 | x |
| BT-474 | −0.072222252 | HR mutant | BRCA2 | Assay 2 | + |
| HCC1599 | 0.117271296 | HR mutant | BRCA2 | Assay 2 | + |
| CAL-51 | 0.547774705 | HR mutant | BRCA2, | Assay 2 | o |
| | | | CHEK1 | | |

### Example 5: Cell Line Derived Xenograft (CDX) Study with HCC1428 Tumors

### Compound B

For this xenograft, supplemental estradiol benzoate injections (40 μg/20 μl/mouse) are administered subcutaneously twice a week to BALB/c nude mice, starting one week prior to cell implantation and continuing through to the end of study. After implantation, HCC1428 tumor were allocated to treatment arms once a median tumor volume of about 181 mm³ was reached. The animals were allocated into 5 treatment groups of 10 mice each according to the groups listed in Table 3A. In Table 3A the abbreviation “p.o.” refers to oral administration and the abbreviation “QD×28” refers to administration once a day for 28 days.

TABLE 3A
Study groups for CDX-HCC1428 treated with Compound B.

| Group | Treatment | Dose regimen | Dose Vol (ml/kg) |
|---|---|---|---|
| 1 | Vehicle: 0.5% | p.o., QD × 28 | 10 |
| | methylcellulose | | |
| | 4000 cPs in | | |
| | distilled water | | |
| 2 | Compound B | 30 mg/kg, p.o., QD × | 10 |
| | | 28 | |
| 3 | Compound B | 100 mg/kg, p.o., QD × | 10 |
| | | 28 | |
| 4 | Compound B | 300 mg/kg, p.o., QD × | 10 |
| | | 28 | |
| 5 | Niraparib | 45 mg/kg, p.o., QD × | 10 |
| | | 28 | |

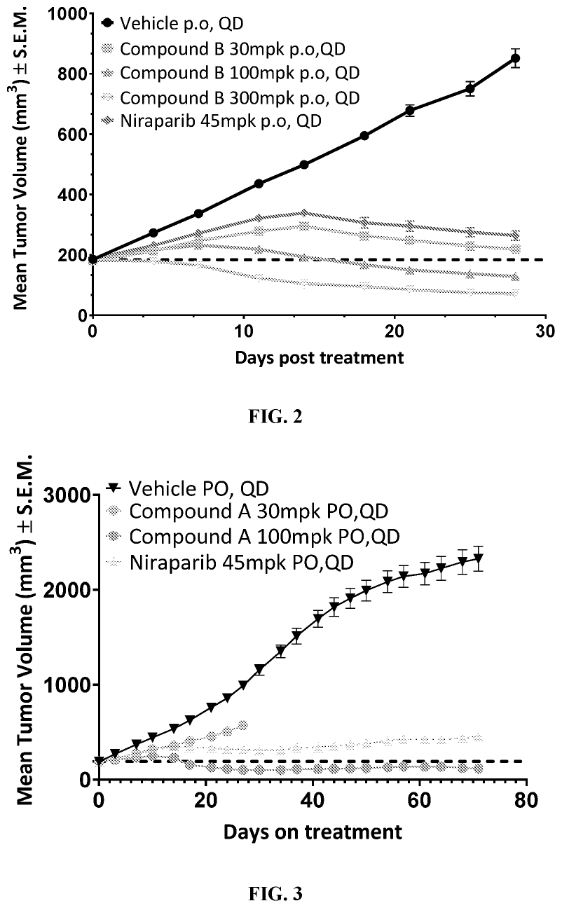

Tumors were measured and mice were weighed twice a week during the experimental period. The tumor volume (mm3) was estimated using the formula: TV=a×b2/2, where “a” and “b” were the long and the short diameter of a tumor, respectively. The Tumor volumes were used to calculate tumor growth inhibition (TGI, an indicator of antitumor activity) using the formula: TGI %: (1−{T/T0/Ct/C0}/1−{C0/Ct})×100 where Tt=median tumor volume of treated at time t, T0=median tumor volume of treated at time 0, Ct=median tumor volume of control at time t and C0=median tumor volume of control at time 0. Animals that received Compound B at dose levels of 30, 100 or 300 mg/kg QD had mean tumor volumes of 165 mm³, 103 mm³ and 50 mm³, respectively, on Day 28 of the treatment The tumor growth inhibition (TGI) of these treatments compared with vehicle group on Day 28 of the treatment is summarized in Table 3B (See FIG. 2).

TABLE 3B
Tumor growth inhibition compared with vehicle group on Day 28.

| | | p value, treated vs. |
|---|---|---|
| Compound B dose | TGI (%) D28 | Vehicle |
| 30 mg/kg | 81.7% | p < 0.001 |
| 100 mg/kg | 89.0% | p < 0.001 |
| 300 mg/kg | 94.6% | p < 0.001 |

Animals that received 45 mg/kg niraparib QD had a mean tumor volume of 230 mm³ (TGI value=75.2%, P<0.001 versus vehicle group). As demonstrated by the present example, in vivo treatment of animals harboring HCC1428 tumors with a PARG inhibitor resulted in tumor growth inhibition as compared to vehicle-treated animals.

### Compound A

For this xenograft, supplemental estradiol benzoate injections (40 μg/20 μl/mouse) are administered subcutaneously twice a week to BALB/c nude mice, starting one week prior to cell implantation and continuing through to the end of study. After implantation, HCC1428 tumor were allocated to treatment arms once a median tumor volume of about 181 mm³ was reached. The animals were allocated into 5 treatment groups of 10 mice each according to the groups listed in Table 4A. In Table 4A the abbreviation “p.o.” refers to oral administration and the abbreviation “QD×28” refers to administration once a day for 28 days.

TABLE 4A
Study groups for CDX-HCC1428 treated with Compound A.

| | | | Dose Vol |
|---|---|---|---|
| Group | Treatment | Dose regimen | (ml/kg) |
| 1 | Vehicle: 0.5% | p.o., QD × 35 | 10 |
| | methylcellulose | | |
| | (400 cPs) + | | |
| | 0.5% Tween 80 in | | |
| | distilled water | | |
| 2 | Compound A | 3 mg/kg, p.o., QD × 28 | 10 |
| 3 | Compound A | 30 mg/kg, p.o., QD × 28 | 10 |
| 4 | Compound A | 100 mg/kg, p.o., QD × 72 | 10 |
| 5 | Niraparib | 45 mg/kg, p.o., QD × 72 | 10 |

Tumors were measured and mice were weighed twice a week during the experimental period. The tumor volume (mm³) was estimated using the formula: TV=a×b2/2, where “a” and “b” were the long and the short diameter of a tumor, respectively. The Tumor volumes were used to calculate tumor growth inhibition (TGI, an indicator of antitumor activity) using the formula: TGI %: (1−{T/T0/Ct/C0}/1−{C0/Ct})×100 where Tt=median tumor volume of treated at time t, T0=median tumor volume of treated at time 0, Ct=median tumor volume of control at time t and C0=median tumor volume of control at time 0. Animals that received Compound A at dose levels of 3 and 30 mg/kg QD had a mean tumor volume of 836 mm³ and 570 mm³ respectively, on Day 28 of the treatment. At 100 mg/kg QD the mean tumor volume achieved was 116 mm3, on Day 71. The tumor growth inhibition (TGI) of these treatments compared with vehicle group on Day 28 or 71 of the treatment is summarized in Table 4B (See FIG. 3).

TABLE 4B
Tumor growth inhibition compared with vehicle group on
Day 28 or 71.

| Compound A | TGI (%) | p value, treated vs. |
|---|---|---|
| dose | D28 or 71 | Vehicle |
| 3 mg/kg | 23% | p < 0.001 |
| 30 mg/kg | 54% | p < 0.001 |
| 100 mg/kg | 103% | p < 0.001 |

Animals that received 45 mg/kg niraparib QD had a mean tumor volume of 230 mm³ (TGI value=75.2%, P<0.001 versus vehicle group). As demonstrated by the present example, in vivo treatment of animals harboring HCC1428 tumors with a PARG inhibitor resulted in tumor growth inhibition as compared to vehicle-treated animals.

### Example 6: Patient-Derived Xenograft (PDX) Study with HBCx 22 and HBCx34 Tumor Models

### HBCx-22 (Compound B)

Female Athymic Nude-Foxn1^{nu} mice with an established growing HBCx-22 tumor between 108 and 288 mm³ were randomized into 4 treatment groups of 10 mice each, according to the groups listed in Table 5A, tumor-bearing mice receive estrogen diluted in drinking water (β-estradiol, 8.5 mg/l), from the date of tumor implant to the end of the study. In Table 5A, the abbreviation “p.o.” refers to oral administration, the abbreviation “QD” refers to administration once a day, and the abbreviation “BID” refers to administration twice a day.

TABLE 5A
HBCx-22 tumor model study groups

| | | | Dose Vol |
|---|---|---|---|
| Group | Treatment | Dose regimen | (ml/kg) |
| 1 | Vehicle | p.o., BID × 41.5 | 10 |
| 2 | Compound B | 30 mg/kg, p.o., QD × 42 | 10 |
| 3 | Compound B | 100 mg/kg, p.o., QD × 42 | 10 |
| 4 | Compound B | 100 mg/kg (i.e. 200 mg/kg/day), | 10 |
| | | p.o., BID × 41.5 | |

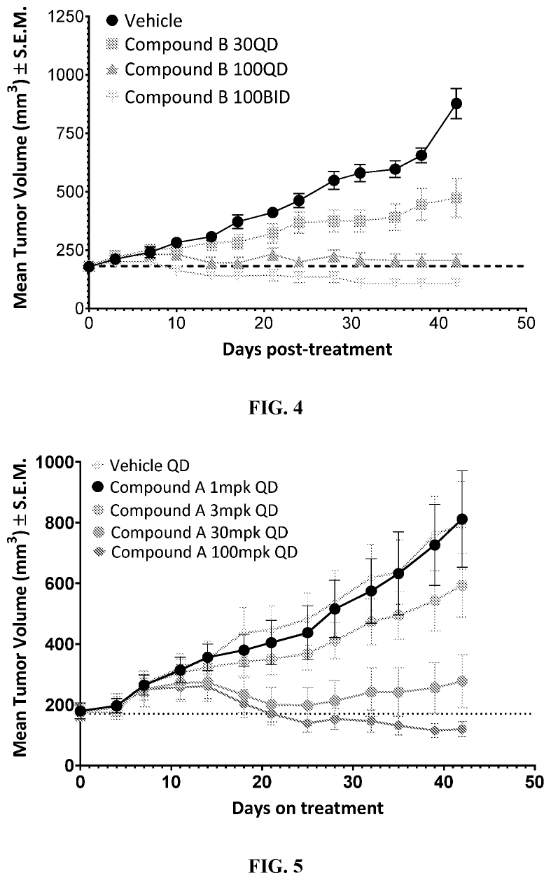

Tumors were measured and mice were weighed twice per week during the experimental period. The tumor volume (mm³) was estimated using the formula: tumor volume (TV)=a×b2/2, where “a” and “b” were the long and the short diameter of a tumor, respectively. The TVs were used to calculate tumor growth inhibition (TGI, an indicator of antitumor activity) using the formula TGI %: (1−{T/T0/Ct/C0}/1−{C0/Ct})×100 where Tt=median tumor volume of treated at time t, T0=median tumor volume of treated at time 0, Ct=median tumor volume of control at time t and C0=median tumor volume of control at time 0. A dose-dependent response to Compound B was demonstrated at 30 and 100 mg/kg, but there was no statistical difference of antitumor efficacy between the groups treated at the dose 100 mg/kg QD or 100 mg/kg BID (FIG. 4). Compound B administered at 30 mg/kg once daily demonstrated statistically significant inhibition of tumor growth, with tumor stabilization for all mice and 1/10 partial regressions. Compound B administered at 100 mg/kg once daily (100 mg/kg/day) demonstrated statistically significant anti-tumor efficacy, with partial tumor regression in 3/10 mice. Compound B administered at 100 mg/kg twice daily (200 mg/kg/day) demonstrated statistically significant anti-tumor efficacy, with partial tumor regression for 6/10 mice as shown in Table 5B. Tumor regression represents tumor volume less than the initial tumor volume on D0.

TABLE 5B
Tumor growth inhibition compared with vehicle group on Day 42.

| Compound B | TGI (%) | p value, treated vs. | Regressions |
|---|---|---|---|
| dose | D42 | Vehicle | (%) |
| 30 mg/kg QD | 58.6 | p < 0.01 | 10 |
| 100 mg/kg QD | 97.3 | p < 0.0001 | 30 |
| 100 mg/kg BID | 110.4 | p < 0.0001 | 60 |

### HBCx-22 (Compound A)

Female Athymic Nude-Foxn1^{nu} mice with an established growing HBCx-22 tumor between 108 and 288 mm³ were randomized into 5 treatment groups of 8 mice each, according to the groups listed in Table 6A, tumor-bearing mice receive estrogen diluted in drinking water (β-estradiol, 8.5 mg/l), from the date of tumor implant to the end of the study. In Table 6A, the abbreviation “p.o.” refers to oral administration, the abbreviation “QD” refers to administration once a day, and the abbreviation “BID” refers to administration twice a day.

TABLE 6A
HBCx-22 tumor model study groups

| | | | Dose Vol |
|---|---|---|---|
| Group | Treatment | Dose regimen | (ml/kg) |
| 1 | Vehicle | p.o., BID × 42 | 10 |
| 2 | Compound A | 3 mg/kg, p.o., QD × 42 | 10 |
| 3 | Compound A | 10 mg/kg, p.o., QD × 42 | 10 |
| 4 | Compound A | 30 mg/kg, p.o., QD × 42 | 10 |
| 5 | Compound A | 100 mg/kg, p.o., QD × 41.5 | 10 |

Tumors were measured and mice were weighed twice per week during the experimental period. The tumor volume (mm³) was estimated using the formula: tumor volume (TV)=a×b2/2, where “a” and “b” were the long and the short diameter of a tumor, respectively. The TVs were used to calculate tumor growth inhibition (TGI, an indicator of antitumor activity) using the formula TGI %: (1−{T/T0/Ct/C0}/1−{C0/Ct})×100 where Tt=median tumor volume of treated at time t, T0=median tumor volume of treated at time 0, Ct=median tumor volume of control at time t and C0=median tumor volume of control at time 0. A dose-dependent response to Compound A was demonstrated in the dose in doses 10 to 100 mg/kg, (FIG. 5). Compound A administered at 3 mg/kg showed no significant difference in its anti-tumor activity compared to control and although 10 mpk showed some tumor growth inhibition it was not statistically significant. Robust and statistically significant tumor growth inhibition was observed starting at a dose of 30 mpk with 4/8 as shown in Table 6B. Tumor regression represents tumor volume less than the initial tumor volume at DO.

TABLE 6B
Tumor growth inhibition compared with vehicle group on Day 42.

| Compound A | TGI (%) | p value, treated vs. | Regressions |
|---|---|---|---|
| dose | D42 | Vehicle | (%) |
| 3 mg/kg QD | 0.1 | ns | 0 |
| 10 mg/kg QD | 31.8 | ns | 0 |
| 30 mg/kg QD | 83.4 | p < 0.01 | 50 |
| 100 mg/kg QD | 108.5 | p < 0.01 | 62 |

### HBCx-34 (Compound B)

Female Athymic Nude-Foxn1^{nu} mice with an established growing HBCx-34 tumor between 108 and 288 mm³ were randomized into 4 treatment groups of 10 mice each, according to the groups listed in Table 7A, tumor-bearing mice receive estrogen diluted in drinking water (β-estradiol, 8.5 mg/l), from the date of tumor implant to the end of the study. In Table 7A, the abbreviation “p.o.” refers to oral administration, the abbreviation “QD” refers to administration once a day, and the abbreviation “BID” refers to administration twice a day.

TABLE 7A
HBCx-34 tumor model study groups

| | | | Dose Vol |
|---|---|---|---|
| Group | Treatment | Dose regimen | (ml/kg) |
| 1 | Vehicle | p.o., BID × 34.5 | 10 |
| 2 | Compound B | 30 mg/kg, p.o., QD × 35 | 10 |
| 3 | Compound B | 100 mg/kg, p.o., QD × 35 | 10 |
| 4 | Compound B | 100 mg/kg (i.e. 200 mg/kg/day), | 10 |
| | | p.o., BID × 34.5 | |

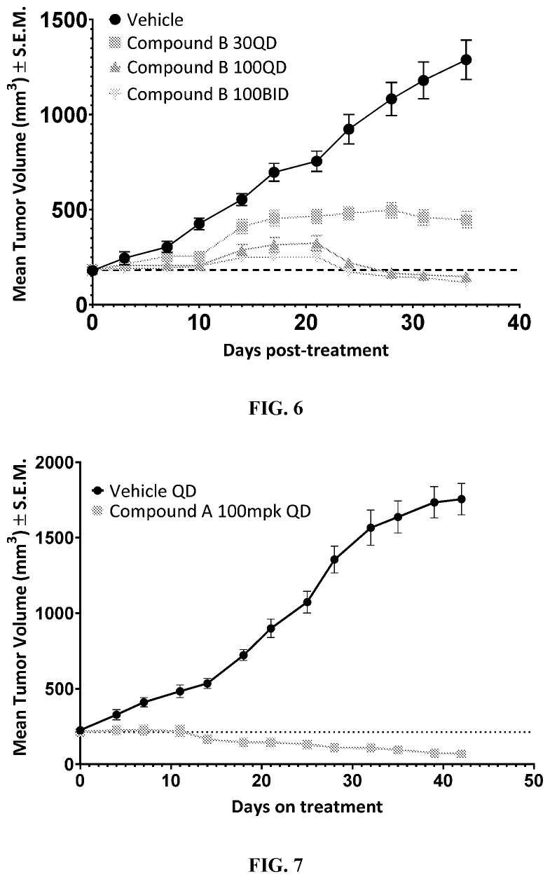

Tumors were measured and mice were weighed twice per week during the experimental period. The tumor volume (mm³) was estimated using the formula: tumor volume (TV)=a×b2/2, where “a” and “b” were the long and the short diameter of a tumor, respectively. The TVs were used to calculate tumor growth inhibition (TGI, an indicator of antitumor activity) using the formula TGI %: (1−{T/T0/Ct/C0}/1−{C0/Ct})×100 where Tt=median tumor volume of treated at time t, T0=median tumor volume of treated at time 0, Ct=median tumor volume of control at time t and C0=median tumor volume of control at time 0. A dose-dependent response to Compound B was demonstrated at 30 and 100 mg/kg, but there was no statistical difference of antitumor efficacy between the groups treated with 100 mg/kg QD or BID (FIG. 6). Compound B administered at 30 mg/kg once daily demonstrated statistically significant inhibition of tumor growth, with tumor stabilization for all mice and 1/10 partial regression. Compound B administered at 100 mg/kg once daily (100 mg/kg/day) demonstrated statistically significant anti-tumor efficacy, with partial tumor regression in 8/10 mice. Compound B administered at 100 mg/kg twice daily (200 mg/kg/day) demonstrated statistically significant anti-tumor efficacy, with partial tumor regression for 8/10 mice as shown on Table 7B. Tumor regression represents tumor volume less than the initial tumor volume on DO.

TABLE 7B
Tumor growth inhibition compared with vehicle group on Day 35.

| Compound B | TGI (%) | p value, treated vs. | Regressions |
|---|---|---|---|
| dose | D35 | Vehicle | (%) |
| 30 mg/kg QD | 77 | p < 0.0001 | 10 |
| 100 mg/kg QD | 103 | p < 0.0001 | 80 |
| 100 mg/kg BID | 106 | p < 0.0001 | 80 |

### HBCx-34 (Compound A)

Female Athymic Nude-Foxn1^{nu} mice with an established growing HBCx-34 tumor between 108 and 288 mm³ were randomized into 2 treatment groups of 8 mice each, according to the groups listed in Table 8A, tumor-bearing mice receive estrogen diluted in drinking water (β-estradiol, 8.5 mg/l), from the date of tumor implant to the end of the study. In Table 8A, the abbreviation “p.o.” refers to oral administration, the abbreviation “QD” refers to administration once a day.

TABLE 8A
HBCx-34 tumor model study groups

| | | | Dose Vol |
|---|---|---|---|
| Group | Treatment | Dose regimen | (ml/kg) |
| 1 | Vehicle | p.o., QD × 45 | 10 |
| 2 | Compound A | 100 mg/kg, p.o., QD × 45 | 10 |

Tumors were measured and mice were weighed twice per week during the experimental period. The tumor volume (mm³) was estimated using the formula: tumor volume (TV)=a×b2/2, where “a” and “b” were the long and the short diameter of a tumor, respectively. The TVs were used to calculate tumor growth inhibition (TGI, an indicator of antitumor activity) using the formula TGI %: (1−{T/T0/Ct/C0}/1−{C0/Ct})×100 where Tt=median tumor volume of treated at time t, T0=median tumor volume of treated at time 0, Ct=median tumor volume of control at time t and C0=median tumor volume of control at time 0. Compound A administered at 100 mg/kg resulted in a robust and statistically significant anti-tumor response, with all mice achieving partial regression as shown in Table 8B (FIG. 7). Tumor regression represents tumor volume less than the initial tumor volume at DO.

TABLE 8B
Tumor growth inhibition compared with vehicle group on Day 45.

| Compound A | TGI (%) | p value, treated vs. | Regressions |
|---|---|---|---|
| dose | D45 | Vehicle | (%) |
| 100 mg/kg QD | 110 | p < 0.0001 | 100 |

### OTHER EMBODIMENTS

Embodiments and implementations have been described. Nevertheless, it will be understood that various modifications may be made without departing from the spirit and scope of the disclosure. Accordingly, other embodiments and implementations are within the scope of the following claims.

1. A Poly(ADP-ribose) glycohydrolase (PARG) modulator for use in a method of treating breast cancer in a patient in need thereof, wherein the patient has a breast cancer tumor and the tumor comprises homologous recombination deficiency (HRD) and is estrogen receptor (ER) positive (ER+), the method comprising administering a therapeutically effective amount of the PARG modulator to the patient in need thereof.

2. A PARG modulator for use according to claim 1, the method comprising:
(a) selecting a patient having a breast cancer tumor wherein the tumor comprises homologous recombination deficiency (HRD) and is estrogen receptor (ER) positive (ER+); and
(b) administering to the patient in need thereof, a therapeutically effective amount of the Poly(ADP-ribose) glycohydrolase (PARG) modulator.

3. The PARG modulator for use according to claim 1 or claim 2, wherein the breast cancer tumor is a human epidermal growth factor receptor-2 (HER2) negative (HER2−) tumor.

4. The PARG modulator for use according to any one of claims 1 to 3, wherein the breast cancer tumor is a progesterone receptor (PR) positive (PR+) tumor.

5. The PARG modulator for use according to any one of claims 1 to 4, wherein the PARG modulator is a small molecule PARG inhibitor.

6. The PARG modulator for use according to any one of claims 1 to 5, wherein the PARG modulator is a compound of Formula (I): 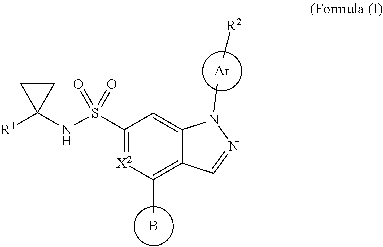
or a pharmaceutically acceptable salt thereof, wherein
R¹ is selected from the group consisting of cyano, C₁₋₂ alkyl, and C₁₋₂ haloalkyl;
Ar is a 5-membered heteroaryl;
X² is CH or CF;
R² is selected from the group consisting of C₁₋₃ alkyl, C₁₋₃ haloalkyl, hydroxyC₁₋₃alkyl, and cyano;
ring B is 5- or 6-membered heterocycloalkyl substituted with R^{a}, R^{b}, and R^{c};
R^{a} is hydrogen, C₁₋₄ alkyl, C₁₋₄ haloalkyl, halo, hydroxy, —C(O)R^{d} (where R^{d} is hydrogen, C₁₋₆ alkyl, or C₁₋₆ haloalkyl); and
R^{b} and R^{c} are independently selected from hydrogen, C₁₋₆ alkyl, hydroxy, C₁₋₆ alkoxy, halo, C₁₋₆ haloalkyl, and C₁₋₆ haloalkoxy.

7. The PARG modulator for use according to claim 6, wherein X² is CH.

8. The PARG modulator for use according to claim 6 or 7, wherein R¹ is cyano.

9. The PARG modulator for use according to claim 6 or 7, wherein R¹ is methyl or ethyl.

10. The PARG modulator for use according to claim 6 or 7, wherein R¹ is methyl.

11. The PARG modulator for use according to any one of claims 6 to 10, wherein Ar is 1,2,4-thiadiazolyl, or 1,3,4-thiadiazolyl.

12. The PARG modulator for use according to any one of claims 6 to 11, wherein Ar is 1,2,4-thiadiazolyl.

13. The PARG modulator for use according to any one of claims 6 to 11, wherein Ar is 1,3,4-thiadiazolyl.

14. The PARG modulator for use according to any one of claims 6 to 13, wherein R² is attached to the carbon atom of Ar that is meta to the atom of Ar that is attached to the nitrogen atom of the remainder of the molecule.

15. The PARG modulator for use according to any one of claims 6 to 14, wherein R² is methyl, ethyl, difluoromethyl, trifluoromethyl, cyano.

16. The PARG modulator for use according to any one of claims 6 to 15, wherein R² is difluoromethyl.

17. The PARG modulator for use according to any one of claims 6 to 16, wherein ring B is morpholinyl, 1,1-dioxothiomorpholinyl, pyrrolidinyl, piperidinyl, 6-oxo-1,6-dihydropyridinyl, or piperazinyl.

18. The PARG modulator for use according to any one of claims 6 to 17, wherein ring B is piperazinyl.

19. The PARG modulator for use according to any one of claims 6 to 18, wherein R^{a} is hydrogen, C₁₋₄ alkyl, C₁₋₄ haloalkyl, halo, hydroxy, —C(O) R^{d} (where R^{d} is hydrogen, C₁₋₆ alkyl, or C₁₋₆ haloalkyl); and R^{b} and R^{c} are independently selected from hydrogen, C₁₋₆ alkyl, hydroxy, C₁₋₆ alkoxy, halo, C₁₋₆ haloalkyl, and C₁₋₆ haloalkoxy.

20. The PARG modulator for use according to any one of claims 6 to 19, wherein R^{a} is hydrogen, C₁₋₄ alkyl, C₁₋₄ haloalkyl, —C(O)R^{d} (where R^{d} is C₁₋₆ alkyl, or C₁₋₆ haloalkyl); and R^{b} and R^{c} are independently selected from hydrogen, C₁₋₆ alkyl, and C₁₋₆ haloalkoxy.

21. The PARG modulator for use according to any one of claims 6 to 20, wherein R^{a} is —C(O)R^{d} where R^{d} is C₁₋₆ alkyl; and R^{b} and R^{c} are independently selected from hydrogen, C₁₋₆ alkyl, and C₁₋₆ haloalkoxy.

22. The PARG modulator for use according to any one of claims 6 to 20, wherein R^{a} is hydrogen C₁₋₄ alkyl, and C₁₋₄ haloalkyl; and R^{b} and R^{c} are independently selected from hydrogen, and C₁₋₆ alkyl.

23. The PARG modulator for use according to any one of claims 6 to 22, wherein the compound is: 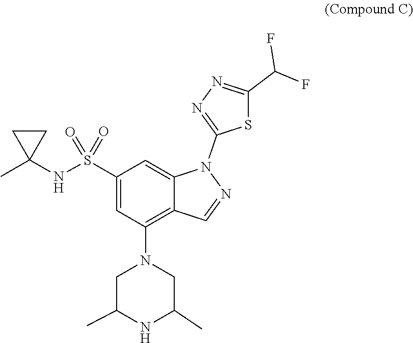 [SMILES: CC1CN(c2cc(S(=O)(=O)NC3(C)CC3)cc3c2cnn3-c2nnc(C(F)F)s2)CC(C)N1]
or a pharmaceutically acceptable salt thereof.

24. The PARG modulator for use according to any one of claims 6 to 22, wherein the compound is: 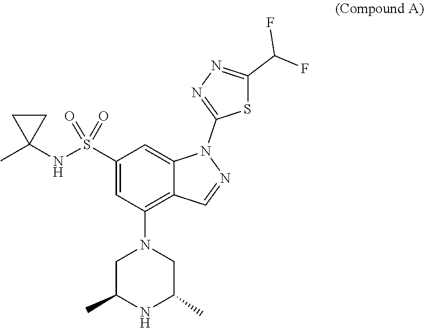 [SMILES: C[C@H]1CN(c2cc(S(=O)(=O)NC3(C)CC3)cc3c2cnn3-c2nnc(C(F)F)s2)C[C@H](C)N1]
or a pharmaceutically acceptable salt thereof.

25. The PARG modulator for use according to any one of claims 6 to 22, wherein the compound is selected from the group consisting of: 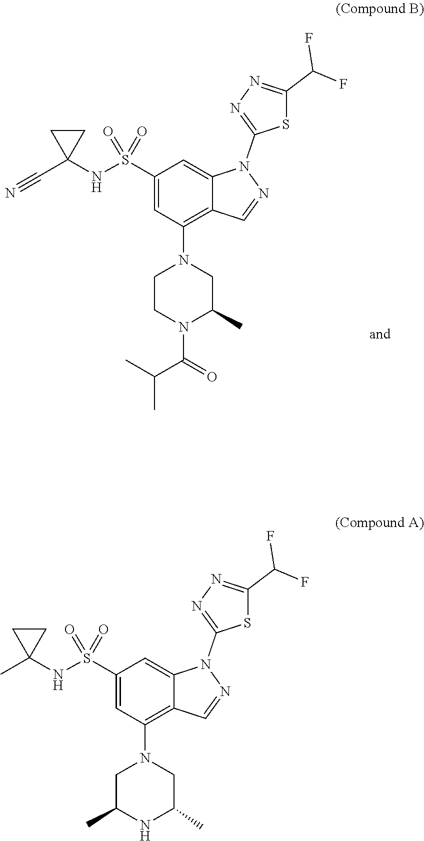 [SMILES: CC(C)C(=O)N1CCN(c2cc(S(=O)(=O)NC3(C#N)CC3)cc3c2cnn3-c2nnc(C(F)F)s2)C[C@H]1C ; C[C@H]1CN(c2cc(S(=O)(=O)NC3(C)CC3)cc3c2cnn3-c2nnc(C(F)F)s2)C[C@H](C)N1]
or a pharmaceutically acceptable salt thereof.

26. The PARG modulator for use according to any one of claims 2 to 25, wherein the selecting of the patient having a breast cancer tumor further comprises obtaining a biological sample from the breast cancer tumor, or obtaining information about a biological sample of the breast cancer tumor.

27. The PARG modulator for use according to claim 26, further comprising assessing the biological sample, or evaluating the information about the biological sample, to determine whether the breast cancer tumor:
(a) comprises homologous recombination deficiency (HRD); and
(b) is ER+.

28. The PARG modulator for use according to claim 27, further comprising assessing the biological sample, or evaluating the information about the biological sample, to determine whether the breast cancer tumor is a HER2− tumor.

29. The PARG modulator for use according to claim 27 or 28, further comprising assessing the biological sample, or evaluating the information about the biological sample, to determine whether the breast cancer tumor is a PR+ tumor.

30. The PARG modulator for use according to any one of claims 26 to 29, further comprising obtaining multiple biological samples, or obtaining information about multiple biological samples, of the breast cancer tumor; wherein the multiple biological samples are taken at separate points of time; and assessing the multiple biological samples, or evaluating the information about multiple biological samples to determine whether the breast cancer tumor:
(a) comprises homologous recombination deficiency (HRD); and
(b) is ER+.

31. The PARG modulator for use according to claim 30, further comprising obtaining multiple biological samples, or obtaining information about multiple biological samples, of the breast cancer tumor; wherein the multiple biological samples are taken at separate points of time; and assessing the multiple biological samples, or evaluating the information about multiple biological samples to determine whether the breast cancer tumor is HER2− tumor.

32. The PARG modulator for use according to claim 30 or 31, further comprising obtaining multiple biological samples, or obtaining information about multiple biological samples, of the breast cancer tumor; wherein the multiple biological samples are taken at separate points of time; and assessing the multiple biological samples, or evaluating the information about multiple biological samples to determine whether the breast cancer tumor is PR+ tumor.

33. The PARG modulator for use according to any one of claims 26 to 32, wherein the biological sample is a biopsy.

34. The PARG modulator for use according to claim 33, wherein the biopsy is a tumor biopsy, or a liquid biopsy.

35. The PARG modulator for use according to any one claims 26 to 34, wherein the biological sample is a tumor biopsy obtained by core needle biopsy.

36. The PARG modulator for use according to any one of claims 26 to 34, wherein the biological sample is a tumor biopsy prepared in the form of tissue sections.

37. The PARG modulator for use according to claim 36, wherein the tissue sections are fixed and paraffin-embedded.

38. The PARG modulator for use according to any one of claims 26 to 37, wherein the biological sample is frozen.

39. The PARG modulator for use according to any one of claims 26 to 38, wherein the biological sample is prepared in the form of isolated cells, lysed cells, homogenate, cell fraction, or a combination thereof.

40. The PARG modulator for use according to claim 39, wherein the biological sample is cultured cells.

41. The PARG modulator for use according to any one of claims 1 to 40, wherein the homologous recombination deficiency (HRD) is characterized by HRD score, HRDetect, single base substitution signature 3 (SBS3), or indel signature 6 (ID6).

42. The PARG modulator for use according to any one of claims 27 to 41, wherein assessing the biological sample or evaluating the information about the biological sample comprises identifying homologous recombination deficiency (HRD) in DNA extracted from a tumor sample.

43. The PARG modulator for use according to any one of claims 27 to 42, wherein assessing the biological sample or evaluating the information about the biological sample comprises identifying homologous recombination deficiency (HRD) in a circulating tumor DNA.

44. The PARG modulator for use according to any one of claims 26 to 42, wherein assessing the biological sample, or evaluating the information about the biological sample comprises determining whether the breast cancer tumor is ER+ tumor.

45. The PARG modulator for use according to any one of claims 26 to 44, wherein assessing the biological sample, or evaluating the information about the biological sample comprises determining whether the breast cancer tumor is HER2− tumor.

46. The PARG modulator for use according to any one of claims 26 to 44, wherein assessing the biological sample, or evaluating the information about the biological sample comprises determining whether the breast cancer tumor is PR+ tumor.

47. The PARG modulator for use according to any one of claims 1 to 46, wherein the PARG modulator is administered to the patient as a monotherapy.

48. The PARG modulator for use according to any one claims 1 to 47, further comprising administering at least one additional therapeutic agent.

49. A method of treating breast cancer in a patient in need thereof, the method comprising:
(a) selecting a patient having a breast cancer tumor wherein the tumor comprises homologous recombination deficiency (HRD) and is estrogen receptor (ER) positive (ER+); and
(b) administering to the patient in need thereof, a therapeutically effective amount of a Poly(ADP-ribose) glycohydrolase (PARG) modulator.

50. Use of a Poly(ADP-ribose) glycohydrolase (PARG) modulator in the manufacture of a medicament for treating breast cancer in a patient in need thereof, wherein the breast cancer in the patient is a tumor and the tumor comprises homologous recombination deficiency (HRD) and is estrogen receptor (ER) positive (ER+).

51. A method of identifying a breast cancer patient susceptible to treatment with a Poly(ADP-ribose) glycohydrolase (PARG) modulator, the method comprising:
(a) identifying a patient having a breast cancer tumor, and
(b) obtaining information about a biological sample of the breast cancer tumor and evaluating the information about the biological sample to determine whether the breast cancer tumor comprises homologous recombination deficiency (HRD) and is estrogen receptor (ER) positive (ER+).

52. The method of claim 51, further comprising evaluating the information about the biological sample to determine whether the breast cancer tumor is a HER2− tumor.

53. The method of claim 51 or 52, further comprising evaluating the information about the biological sample to determine whether the breast cancer tumor is a PR+ tumor.
